SEQUENCE LISTING (14 sequences)

SEQ ID NO: 1 - DNA, 20 bp, synthetic construct
  source 1..20: mol_type = other DNA
ccactttctcctcatttctg

SEQ ID NO: 2 - DNA, 22 bp, synthetic construct
  source 1..22: mol_type = other DNA
ctagctcttcagttgtagtaac

SEQ ID NO: 3 - DNA, 20 bp, synthetic construct
  source 1..20: mol_type = other DNA
gatctgtatctgccacaatg

SEQ ID NO: 4 - DNA, 20 bp, synthetic construct
  source 1..20: mol_type = other DNA
tggtgatacgtattcttccg

SEQ ID NO: 5 - DNA, 21 bp, synthetic construct
  source 1..21: mol_type = other DNA
gaggaaaacattaagaagggc

SEQ ID NO: 6 - DNA, 20 bp, synthetic construct
  source 1..20: mol_type = other DNA
agaaagctcaaacttgacag

SEQ ID NO: 7 - DNA, 20 bp, synthetic construct
  source 1..20: mol_type = other DNA
cctataggattggagacacg

SEQ ID NO: 8 - DNA, 20 bp, synthetic construct
  source 1..20: mol_type = other DNA
gttggtaaatagctgttcgg

SEQ ID NO: 9 - DNA, 21 bp, synthetic construct
  source 1..21: mol_type = other DNA
gagactcagaaaaaggtcatc

SEQ ID NO: 10 - DNA, 21 bp, synthetic construct
  source 1..21: mol_type = other DNA
gtcttcaaagaactcagatgc

SEQ ID NO: 11 - DNA, 21 bp, synthetic construct
  source 1..21: mol_type = other DNA
ctagttcctgcatccattttg

SEQ ID NO: 12 - DNA, 20 bp, synthetic construct
  source 1..20: mol_type = other DNA
atatttgagacattgctggc

SEQ ID NO: 13 - DNA, 20 bp, synthetic construct
  source 1..20: mol_type = other DNA
gatgtatgaaggctttggtc

SEQ ID NO: 14 - DNA, 20 bp, synthetic construct
  source 1..20: mol_type = other DNA
tgtgcacttttattggtctc

(19) United States
(12) Patent Application Publication
(10) Pub. No.: US 2025/0352528 A1
(43) Pub. Date: Nov. 20, 2025

(54) TOPICAL ROSIGLITAZONE FOR RADIATION-INDUCED SKIN INJURY

(71) Applicant: The Brigham and Women's Hospital, Inc., Boston, MA (US)

(72) Inventor: Shailesh Agarwal, Boston, MA (US)

(21) Appl. No.: 18/872,403

(22) PCT Filed: Jun. 7, 2023
(86) PCT No.: PCT/US2023/068068

§ 371 Date: Dec. 6, 2024

Related U.S. Application Data
(60) Provisional application No. 63/349,891, filed on Jun. 7, 2022.
(60) Provisional application No. 63/404,249, filed on Sep. 7, 2022.

(51) Int. Cl. A61K 31/4439; A61K 31/427; A61P 7/10

(52) U.S. Cl. CPC A61K 31/4439; A61K 31/427; A61P 7/10

(57) ABSTRACT

While primary lymphedema is rare, numerous cancer patients develop secondary lymphedema, or the retention of lymphatic fluid. Lymphedema is characterized by progressive, irreversible fibroadipose tissue deposition. Non-surgical approaches such as compression therapy are the most common strategies to address lymphedema but are inadequate in the long term. Surgical procedures can reconstitute lymphatic drainage, but this approach is not curative. A new approach is needed to mitigate fibroadipose tissue deposition in lymphedema.

### CROSS-REFERENCE TO RELATED APPLICATIONS

The present application claims priority to U.S. Provisional Patent Application No. 63/349,891, filed Jun. 7, 2022, and to U.S. Provisional Patent Application No. 63/404,249, filed Sep. 7, 2022, the entire contents of which are hereby incorporated by reference.

### STATEMENT REGARDING FEDERALLY SPONSORED RESEARCH

Not Applicable.

### REFERENCE TO A SEQUENCE LISTING

A Sequence Listing accompanies this application and is submitted as an xml file of the sequence listing named “129319_00939_sequences.xml” which is 13,424 bytes in size and was created on Jun. 5, 2023. The sequence listing is electronically submitted via Patent Center and is incorporated herein by reference in its entirety.

### FIELD OF THE INVENTION

The disclosed technology is generally directed to a method of treatment of lymphedema. More particularly the technology is directed to a method of treatment of lymphedema using a peroxisome proliferator-activated receptor gamma agonist (PPARg agonist).

### BACKGROUND

Primary lymphedema is idiopathic and results from an error in lymphatic development. Secondary lymphedema is an acquired condition that can be caused by damage to a normally developed lymphatic system, often due to an infection, injury, cancer treatment, inflammation of the limb or a lack of limb movement. It is characterized by limb swelling due to impaired drainage of lymphatic fluid. Patients experience loss of range of motion, feelings of heaviness, and hardening/thickening of the skin. In late-stage lymphedema the skin becomes susceptible to deep, poorly healing wounds and is in danger of infection. Overall, patients report reduced physical, functional, social, and emotional well-being. In the United States, this condition affects over 5 million individuals who have undergone removal of lymph nodes in the affected extremity for cancer surgery.

Currently there is no cure for lymphedema (either primary or secondary) and treatment focuses on reducing the swelling and preventing complications. Non-surgical approaches include compression or manual lymphatic drainage; however, these treatments are painful and time-consuming. Surgical procedures to reconstitute lymphatic drainage are not curative and patients still require continued long-term compression.

Patients with head/neck cancer often receive radiation, which can result in soft tissue injury. For example, radiation to the neck may cause damage to the vessels, nerves, muscles, skin, and lymphatic system. Damage to the lymphatic system may result in lymphedema and the skin in the irradiated area may become thin. Complex reconstructive operations demand long operative hours, technical expertise, and access to specialized operating supplies. Oral complications caused by radiation therapy to the head and neck include fibrosis in the mucous membrane in the mouth.

As a result, strategies to alleviate secondary lymphedema and radiation-induced tissue injury are needed to improve outcomes for cancer survivors after head/neck radiation.

### BRIEF SUMMARY

The present disclosure meets the foregoing needs by providing a method of treatment of lymphedema using a peroxisome proliferator-activated receptor gamma agonist (PPARg agonist).

In one aspect, the invention provides a method for treating and/or preventing lymphedema in a subject, the method comprising administering to a subject in need of treatment and/or prevention of lymphedema an effective amount of a peroxisome proliferator-activated receptor gamma agonist (PPARg agonist) or a pharmaceutically acceptable salt thereof. In one embodiment of the method, the PPARg agonist is a thiazolidinedione or a pharmaceutically acceptable salt thereof. In one embodiment of the method, the thiazolidinedione is troglitazone, rosiglitazone or pioglitazone. In one embodiment of the method, the effective amount of the PPARg agonist is administered systemically. In one embodiment, the method further comprises co-administration of compression therapy. In one embodiment of the method, the effective amount of the PPARg agonist is administered systemically.

In another aspect, the invention provides a method for reducing the total amount of fibroadipose tissue in a subdermal layer in a subject, the method comprising administering to the subject in need of reducing the total amount of fibroadipose tissue in the subdermal layer an effective amount of a PPARg agonist, wherein the PPARg agonist reduces fibrosis, reduces the number of adipocytes, and reduces the size of adipocytes. In one embodiment of the method, the PPARg agonist is a thiazolidinedione, or a pharmaceutically acceptable salt thereof. In one embodiment of the method, the thiazolidinedione is troglitazone, rosiglitazone, pioglitazone, or a pharmaceutically acceptable salt thereof. In one embodiment of the method, the effective amount of the PPARg agonist is administered systemically.

In yet another aspect, the invention provides a method for rescuing adipogenic gene expression in a subject, the method comprising administering to the subject in need of rescuing adipogenic gene expression an effective amount of a PPARg agonist. In one embodiment of the method, the adipogenic gene expression was reduced among cells which have been exposed to tumor necrosis factor alpha (TNFα). In one embodiment of the method, the PPARg agonist is a thiazolidinedione. In one embodiment of the method, the thiazolidinedione is troglitazone, rosiglitazone or pioglitazone. In one embodiment of the method, the effective amount of the PPARg agonist is administered systemically.

In still another aspect, the invention provides a method for reducing fibrogenic gene expression in a subject, the method comprising administering to the subject in need of reduced fibrogenic gene expression an effective amount of a PPARg agonist. The method of claim 16, wherein the PPARg agonist reduces fibrogenic gene expression by cells which have been exposed to transforming growth factor-beta 1 (TGFβ1). In one embodiment of the method, the PPARg agonist is a thiazolidinedione. In one embodiment of the method, the thiazolidinedione is troglitazone, rosiglitazone or pioglitazone. In one embodiment of the method, the effective amount of the PPARg agonist is administered systemically. In one embodiment of the method, the PPARg agonist is rosiglitazone administered orally at 4 mg/day, 5 mg/day, 6 mg/day, 7 mg/day or 8 mg/day.

In yet another aspect, the invention provides a method for treating and/or preventing a radiation-induced skin tissue injury in a subject undergoing or having received radiation therapy, the method comprising administering topically to skin tissue of a subject in need of treatment and/or prevention of a radiation-induced skin tissue injury an effective amount of a PPARg agonist. In one embodiment of the method, the radiation-induced tissue injury is atrophy, fibrosis, or tissue loss, partial-thickness skin loss, full-thickness skin loss, ulceration or any combination thereof. In one embodiment of the method, the PPARg agonist is a thiazolidinedione. In one embodiment of the method, the thiazolidinedione is troglitazone, rosiglitazone or pioglitazone. In one embodiment of the method, the rosiglitazone is administered topically as a mixture with a carrier, where the weight ratio of rosiglitazone to carrier is in a range of about 1:5,000 to about 1:20,000.

These and other features, aspects, and advantages of the present invention will become better understood upon consideration of the following detailed description, drawings and appended claims.

### BRIEF DESCRIPTION OF THE DRAWINGS

Non-limiting embodiments of the present invention will be described by way of example with reference to the accompanying Figures, which are schematic and are not intended to be drawn to scale. In the Figures, each identical or nearly identical component illustrated is typically represented by a single numeral. For purposes of clarity, not every component is labeled in every Figure, nor is every component of each embodiment of the invention shown where illustration is not necessary to allow those of ordinary skill in the art to understand the invention.

FIG. 1 shows a mouse model of hindlimb lymphedema recapitulates augmented fibrosis and adipose tissue deposition observed in patients with lymphedema. Panel (A) Clinical image of affected and unaffected extremity in patient with secondary lymphedema, magnetic resonance imaging (MRI) showing subcutaneous fibroadipose tissue, and lipoaspirate obtained from this patient; Panel (B) Surgical procedure for hindlimb lymphadenectomy in mice; Panel (C) Normalized circumference of hindlimb after lymphadenectomy relative to uninjured hindlimb. Panel (D) Normalized edema size of hindlimb after lymphadenectomy relative to uninjured hindlimb. Panel (E) H&E staining of control (uninjured), lymphedema day 7, and lymphedema day 21 hindlimbs, magnification 4×, scale bar: 500 μm. Panel (F) Picrosirius red staining of control (uninjured), lymphedema day 7, and lymphedema day 21 hindlimbs, magnification 4×, scale bar: 500 μm. Panel (G) Normalized quantification of hindlimb fibrosis; Panel (H) Quantification of overall hindlimb skin thickness; Panel (I) Quantification of hindlimb fibroadipose tissue thickness; and Panel (J) Quantification of hindlimb dermal thickness. Statistical significance is indicated by *p<0.05, **p<0.01, ***p<0.001, ****p<0.0001. Abbreviations: D, dermis; F, fibroadipose tissue; M, muscle.

FIG. 2 shows treatment effect of rosiglitazone on hindlimb lymphedema in a mouse model. Panel (A) Immunostaining of control (uninjured) and lymphedema day 7 sections for pSmad 2/3, magnification 20×, scale bar: 100 μm. Panel (B) Expression levels of adipogenic genes among adipose-derived mesenchymal cells treated with TGFβ1 with or without rosiglitazone; Panel (C) Expression levels of fibrogenic genes among adipose-derived mesenchymal cells treated with TGFβ1 with or without rosiglitazone; Panel (D) Normalized circumference of hindlimbs after lymphadenectomy with or without rosiglitazone treatment; Panel (E) Normalized edema size of hindlimbs after lymphadenectomy with or without rosiglitazone treatment. Panel (F) H&E staining of hindlimbs after lymphadenectomy with or without rosiglitazone, magnification 4×, scale bar: 500 μm. Panel (G) Picrosirius red staining of hindlimbs after lymphadenectomy with or without rosiglitazone, magnification 4×, scale bar: 500 μm; Panel (H) Normalized quantification of hindlimb fibrosis; Panel (I) Quantification of hindlimb skin thickness; Panel (J) Quantification of hindlimb fibroadipose tissue thickness; and Panel (K) Quantification of hindlimb dermal thickness. Statistical significance is indicated by *p<0.05, **p<0.01, ***p<0.001, ****p<0.0001. Abbreviations: D, dermis; F, fibroadipose tissue; M, muscle.

FIG. 3 shows lymphedema modifies the presence of fate of PDGFRα+ cells. Panel (A) Immunostaining for PDGFRα and Ki67 in control (uninjured) and lymphedema day 7 tissue, magnification 20×, scale bar: 100 μm; Panel (B) Representative flow cytometry plots showing detection of PDGFRα+ in hindlimb tissue; Panel (C) Quantification of PDGFRα+ cells in 1 cm×1 cm area of skin from control and lymphedema day 7 hindlimbs; Panel (D) Representative flow cytometry plots showing detection of PDGFRα+ adipocytes (PDGFRα+LipidTOX+); Panel (E) Quantification of PDGFRα+LipidTOX+ cells in 1 cm×1 cm area of skin from control and lymphedema day 7 hindlimbs; Panel (F) Representative flow cytometry plot showing cell size (FSC-A) among PDGFRα+LipidTOX+ cells; Panel (G) Quantification of FSChigh and FSClow PDGFRα+LipidTOX+ cells in 1 cm×1 cm area of skin from control and lymphedema day 7 hindlimbs; and Panel (H) Histogram showing size distribution of PDGFRα+LipidTOX+ cells measured by FSC-A.

FIG. 4 shows rosiglitazone reduces presence and size of PDGFRα+ adipocytes after lymphadenectomy. Panel (A) Immunostaining for PDGFRα and Ki67 in untreated and rosiglitazone-treated hindlimbs after lymphadenectomy, magnification 20×, scale bar: 100 μm; Panel (B) Representative flow cytometry plots showing detection of PDGFRα+ in hindlimb tissue after lymphadenectomy with or without rosiglitazone; Panel (C) Quantification of PDGFRα+ cells in 1 cm×1 cm area of skin from untreated and rosiglitazone-treated hindlimbs after lymphadenectomy; Panel (D) Representative flow cytometry plots showing detection of PDGFRα+ adipocytes (PDGFRα+LipidTOX+) in untreated or rosiglitazone-treated hindlimbs after lymphadenectomy; Panel (E) Quantification of PDGFRα+LipidTOX+ cells in 1 cm×1 cm area of skin from untreated and rosiglitazone-treated hindlimbs after lymphadenectomy; Panel (F) Representative flow cytometry plot showing cell size (FSC-A) among PDGFRα+LipidTOX+ cells from untreated and rosiglitazone-treated hindlimbs after lymphadenectomy; Panel (G) Quantification of FSChigh and FSClow PDGFRα+LipidTOX+ cells in 1 cm×1 cm area of skin from untreated and rosiglitazone-treated hindlimbs after lymphadenectomy; and Panel (H) Histogram showing size distribution of PDGFRα+LipidTOX+ cells measured by FSC-A in untreated and rosiglitazone-treated hindlimbs after lymphadenectomy. Statistical significance is indicated by *p<0.05, **p<0.01, ***p<0.001. Abbreviations: D, dermis; F, fibroadipose tissue; M, muscle.

FIG. 5 shows increased presence of CD4+ T-cells in the hindlimb after lymphadenectomy. Panel (A) Representative flow cytometry plots identifying CD4+ cells in the hindlimb; Panel (B) Quantification of CD4+ cells 1 cm×1 cm area of skin from hindlimbs with or without lymphadenectomy; and Panel (C) Immunostaining for CD4 and CD8 in uninjured and lymphedema day 7, magnification 20×, scale bar: 100 μm. Statistical significance is indicated by **p<0.01. Abbreviations: D, dermis; F, fibroadipose tissue; M, muscle.

FIG. 6. shows effect of rosiglitazone on expression of adipogenic genes among TNFα-treated adipose-derived mesenchymal stem cells (AdMSCs).

FIG. 7 shows PPARg production is increased in lymphedema and maintained after rosiglitazone treatment. Magnification 20×, scale bar: 100 μm. Abbreviations: D, dermis; F, fibroadipose tissue; M, muscle.

FIG. 8 shows the proposal strategy in Example 2 for control experiment and testing topical rosiglitazone Panel (A) A control experiment includes histology, flow cytometry, and bulk RNA sequencing following a radiation treatment of mouse or human scalp tissue. Panel (B) Mouse scalp is treated with radiation and topical rosiglitazone, followed by serial photography, histology, and bulk RNA sequencing, to demonstrate the effects of rosiglitazone.

FIG. 9 shows photographs of Panel (A) radiated human scalp and Panel (B) non-radiated human scalp.

FIG. 10 shows histological findings in Panel (A) radiated human scalp and Panel (B) non-radiated human scalp.

FIG. 11 shows reduced adipogenic gene expression in vitro after irradiation.

FIG. 12 shows histology of inguinal fat Panels (A, B) and skin Panels (C, D) from adipocyte-lineage tracing mice after ex vivo radiation Panels (A, C), without radiation Panels (B, D) and tissue culture.

FIG. 13 shows the histology of skin from adipocyte-lineage tracing mice after ex vivo radiation and tissue culture with and without rosiglitazone.

### DETAILED DESCRIPTION

Currently there is no cure or effective treatment for lymphedema, where late-stage lymphedema can include drastic skin changes such as fat deposits and fibrosis, or skin thickening. Patients receiving radiation for head/neck cancer often receive radiation treatments which can result in skin thinning and lymphedema. Strategies to reduce or eliminate lymphedema as well as radiation-induced tissue injury are needed to improve quality of life and outcomes for cancer survivors. The strategies for treating lymphedema as disclosed herein may be applied to either primary lymphedema or secondary lymphedema.

The cellular mediators responsible for the deposition of fibroadipose tissue are poorly understood and may serve as a therapeutic target. As disclosed herein, we present a strategy to reduces fibroadipose tissue deposition in lymphedema by treatment with a PPARg agonist. For example, reduced fibrosis and fibrogenic signaling is observed in tissues treated with the PPARg agonist rosiglitazone. Furthermore, treatment of tissues with rosiglitazone results in a reduced number of total cells capable of adipogenic differentiation, and a reduction in the size of adipocytes.

Methods for treating subjects with the compounds disclosed herein are provided. Suitably, the methods for treating a subject comprise administering to the subject an effective amount of one or more PPARg agonist or a pharmaceutical composition comprising the effective amount of one or more PPARg agonists. As used herein, a “subject” may be interchangeable with “patient” or “individual” and means an animal, which may be a human or non-human animal, in need of treatment. In particular embodiments, the subject is a human subject.

As used herein, the terms “treating” or “to treat” each mean to alleviate symptoms, eliminate the causation of resultant symptoms either on a temporary or permanent basis and/or to prevent or slow the appearance or to reverse the progression or severity of resultant symptoms of the named disease or disorder. As such, the methods disclosed herein encompass both therapeutic and prophylactic administration. In some embodiments, the subject is responsive to therapy with one or more of the compounds disclosed herein in combination with one or more additional therapeutic agents.

According to an aspect as disclosed herein, a method for treating and/or preventing lymphedema in a subject can include administering to a subject in need of treatment and/or prevention of lymphedema an effective amount of a PPARg agonist or a pharmaceutically acceptable salt thereof. In a particular embodiment, the method of treating and/or preventing lymphedema in a subject can include administering to a subject in need of treatment and/or prevention of secondary lymphedema an effective amount of a PPARg agonist or a pharmaceutically acceptable salt thereof.

The PPARg agonist can be a thiazolidinedione or a pharmaceutically acceptable salt thereof. In some embodiments, the thiazolidinedione can be troglitazone, rosiglitazone or pioglitazone, or any pharmaceutically acceptable salts thereof.

The PPARg agonist can be administered systemically as a pharmaceutical composition. Pharmaceutical compositions comprising the PPARg agonist may be adapted for administration by any appropriate route, for example by the oral (including buccal or sublingual), rectal, nasal, topical (including buccal, sublingual or transdermal), vaginal or parenteral (including subcutaneous, intramuscular, intravenous or intradermal) route. Such formulations may be prepared by any method known in the art of pharmacy, for example by bringing into association the active ingredient with the carrier(s) or excipient(s).

Pharmaceutical compositions adapted for oral administration may be presented as discrete units such as capsules or tablets; powders or granules; solutions or suspensions in aqueous or non-aqueous liquids; edible foams or whips; or oil-in-water liquid emulsions or water-in-oil liquid emulsions.

As used herein, the term “dermal” means of or relating to the skin and is used interchangeably herein with “cutaneous.” As used herein, “transdermal” means across the skin to the subcutaneous tissues and, often, into the systemic vascular or lymphatic circulation. The term “topical” as used herein means pertaining to the skin. Thus, when a composition is applied topically, it is applied to the skin. It will be understood by those of ordinary skill in the art, however, that the term “topical” does not necessarily refer to where the composition will remain, but rather how it is applied.

Pharmaceutical compositions of the PPARg agonist adapted for transdermal administration may be presented as discrete patches intended to remain in intimate contact with the epidermis of the recipient for a prolonged period of time. For example, the active ingredient may be delivered from the patch by iontophoresis.

Pharmaceutical compositions adapted for topical administration may be formulated as ointments, creams, suspensions, lotions, powders, solutions, pastes, gels, impregnated dressings, sprays, aerosols or oils and may contain appropriate conventional additives such as preservatives, solvents to assist drug penetration and emollients in ointments and creams. Topical administration of the PPARg agonist can include nanosystems or drug-loaded particles characterized by different morphologies, such as nanocapsules, nanospheres, liposomes, foams, carbon nanotube, dendrimers, cubosomes, niosomes, and hydrogels.

For applications to the eye or other external tissues, for example the mouth and skin, the pharmaceutical compositions are preferably applied as a topical ointment or cream. When formulated in an ointment, the PPARg agonist may be employed with either a paraffinic or a water-miscible ointment base. Alternatively, the PPARg agonist may be formulated in a cream with an oil-in-water cream base or a water-in-oil base. Pharmaceutical compositions adapted for topical administration to the eye include eye drops where the active ingredient is dissolved or suspended in a suitable carrier, especially an aqueous solvent. Pharmaceutical compositions adapted for topical administration in the mouth include lozenges, pastilles and mouth washes.

Pharmaceutical compositions adapted for parenteral administration include aqueous and non-aqueous sterile injection solutions which may contain antioxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example sealed ampoules and vials, and may be stored in a freeze-dried (lyophilized) condition requiring only the addition of the sterile liquid carrier, for example water for injections, immediately prior to use. Extemporaneous injection solutions and suspensions may be prepared from sterile powders, granules and tablets.

As used herein the term “effective amount” refers to the amount or dose of the compound that provides the desired effect. In some embodiments, the effective amount is the amount or dose of the compound, upon single or multiple dose administration to the subject, which provides the desired effect in the subject under diagnosis or treatment. The PPARg agonist may be administered to a subject in an effective amount in such that the intracellular receptor class of the peroxisome proliferator-activated receptors, specifically PPARg, is activated. Suitably, the desired effect may be slowing progression of lymphedema, halting lymphedema, reversing lymphedema, reducing lymphedema, or improving lymph drainage. In some embodiments, the effective amount of the PPARg agonist results in slowing lymphedema, reducing lymphedema, or improving lymph fluid flow.

An effective amount can be readily determined by those of skill in the art, including an attending diagnostician, by the use of known techniques and by observing results obtained under analogous circumstances. In determining the effective amount or dose of compound administered, a number of factors can be considered by the attending diagnostician, such as: the species of the subject; its size, age, and general health; the degree of involvement or the severity of the disease or disorder involved; the response of the individual subject; the particular compound administered, the mode of administration; the bioavailability characteristics of the preparation administered; the dose regimen selected; the use of concomitant medication; and other relevant circumstances.

A “subject in need of treatment” may include a subject having a disease, disorder, or condition that may be characterized by lymphedema or a disease associated with or that contributes to the development or progression of lymphedema.

In an embodiment, the method of treating lymphedema by administration of a PPARg agonist can include co-administration of compression therapy. Compression therapy can include use of compression bandages such as low-stretch bandages. Compression bandages are used to wrap the entire limb to encourage lymph fluid to flow back toward the trunk of the body. Compression therapy can include use of compression garments. Compression garments are close-fitting elastic sleeves or stockings that compress the limb to encourage lymph fluid drainage. Compression therapy can include use of sequential pneumatic compression where a sleeve worn over the affected limb is connected to a pump that intermittently inflates the sleeve, putting pressure on the limb and moving lymph fluid away from the fingers or toes.

In some embodiments, the method of treating lymphedema can include administration of the PPARg agonist along with one or more surgical procedures for lymphedema. In some embodiments, the surgical procedure can include a lymph node transplant. In this procedure, lymph nodes are taken from a different area of the body and then attached to the network of lymph vessels in the affected limb. In some embodiments, the surgical procedure can include a creating new drainage paths to make new connections between the lymph network and blood vessels. The excess lymph fluid is then removed from the limb via blood vessels. In some embodiments, the surgical procedure can include removal of fibrous tissue. In severe lymphedema, the soft tissues in the limb become fibrous and hardened. Removing some of this hardened tissue, often through liposuction, can improve the limb's function. In very severe cases, hardened tissue and skin may be removed with a scalpel. Administration of the PPARg agonist may be used in combination with any of these surgical procedures or non-surgical treatments to treat lymphedema.

According to an aspect as disclosed herein, a method for reducing the total amount of fibroadipose tissue in a subdermal layer in a subject afflicted with lymphedema can include administering to the subject in need of reducing the total amount of fibroadipose tissue in the subdermal layer an effective amount of a PPARg agonist. In an embodiment, administration of the PPARg agonist reduces fibrosis in a subdermal layer in a subject afflicted with lymphedema. In another embodiment, administration of the PPARg agonist reduces the number of adipocytes in the subdermal layer. In yet another embodiment, administration of the PPARg agonist in the subdermal layer. In a particular embodiment, administration of the PPARg agonist reduces fibrosis, reduces the number of adipocytes, and reduces the size of adipocytes in the subdermal layer.

According to an aspect as disclosed herein, a method for rescuing adipogenic gene expression in a subject can include administering to the subject an effective amount of a PPARg agonist. Adipogenic gene expression can be reduced among cells which have been exposed to tumor necrosis factor alpha (TNFα). TNFα is present during the development and progression of secondary lymphedema. In some embodiments, the PPARg agonist revives adipogenic gene expression by adipose-derived mesenchymal cells. The adipogenic genes whose expression can be rescued by the PPARg agonist can include Adipoq and Lpl.

According to an aspect as disclosed herein, a method for reducing fibrogenic gene expression in a subject can include administering to the subject in need of reduced fibrogenic gene expression an effective amount of a PPARg agonist. The PPARg agonist reduces fibrogenic gene expression by cells which have been exposed to transforming growth factor-beta 1 (TGFβ1). TGFβ1 is present during the development and progression of secondary lymphedema. In some embodiments, the PPARg agonist reduces fibrogenic gene expression by adipose-derived mesenchymal cells. The fibrogenic genes can include Col1a1, which encodes for collagen, Ctgf, which encodes for a connective tissue growth factor, Fn1, which encodes for fibronectin, a high-molecular weight glycoprotein of the extracellular matrix, and Actb, which encodes for β-actin, a protein that form microfilaments in the cytoskeleton.

According to an aspect as disclosed herein, a method for treating and/or preventing a radiation-induced skin tissue injury in a subject undergoing or having received radiation therapy, can include administering topically to skin tissue of the subject an effective amount of a PPARg agonist.

The radiation-induced tissue injury can be atrophy, fibrosis, or tissue loss, partial-thickness skin loss, full-thickness skin loss, ulceration or any combination thereof. The skin tissue can be any skin tissue of the head or neck, including skin tissue of the scalp, face, cheeks, eyes, eyelids, nose, mouth, ears, or neck. The skin tissue can include skin tissue inside the mouth or throat, the tongue, or inside the nose.

The PPARg agonist can be a thiazolidinedione, or any pharmaceutically acceptable salt thereof. In some embodiments, the thiazolidinedione is troglitazone, rosiglitazone or pioglitazone. The PPARg can be administered topically. In a particular embodiment, the PPARg agonist is rosiglitazone. In an embodiment, troglitazone can be topically administered as a mixture with a carrier. The weight ratio of troglitazone to carrier can be about 1:1,000, about 1:5,000, about 1:10,000, about 1:50,000, or about 1:100,000. In an embodiment, rosiglitazone can be topically administered as a mixture with a carrier. The weight ratio of rosiglitazone to carrier can be about 1:1,000, about 1:5,000, about 1:10,000, about 1:50,000, or about 1:100,000. In an embodiment, pioglitazone can be topically administered as a mixture with a carrier. The weight ratio of pioglitazone to carrier can be about 1:1,000, about 1:5,000, about 1:10,000, about 1:50,000, or about 1:100,000.

In an embodiment, the PPARg agonist is administered orally in an effective amount. In a particular embodiment, rosiglitazone can be administered orally at 4 mg/day, 5 mg/day, 6 mg/day, 7 mg/day or 8 mg/day. In another embodiment, the PPARg agonist is troglitazone administered orally at 5 mg/day, 10 mg/day, 20 mg/day, 50 mg/day, 100 mg/day, 200 mg/day, 300 mg/day, 400 mg/day, 500 mg, day, 600 mg, day, 700 mg/day, or 800 mg/day. In an embodiment, the PPARg agonist is pioglitazone administered orally at 5 mg/day, 10 mg/day, 15 mg/day, 20 mg/day, 25 mg/day, 30 mg/day, 35 mg/day, 40 mg/day, 45 mg/day, 50 mg/day, 55 mg/day, or 60 mg/day.

### Miscellaneous

Unless otherwise specified or indicated by context, the terms “a”, “an”, and “the” mean “one or more.” For example, “a molecule” should be interpreted to mean “one or more molecules.”

As used herein, “about”, “approximately,” “substantially,” and “significantly” will be understood by persons of ordinary skill in the art and will vary to some extent on the context in which they are used. If there are uses of the term which are not clear to persons of ordinary skill in the art given the context in which it is used. “about” and “approximately” will mean plus or minus≤10% of the particular term and “substantially” and “significantly” will mean plus or minus>10% of the particular term.

As used herein, the terms “include” and “including” have the same meaning as the terms “comprise” and “comprising.” The terms “comprise” and “comprising” should be interpreted as being “open” transitional terms that permit the inclusion of additional components further to those components recited in the claims. The terms “consist” and “consisting of” should be interpreted as being “closed” transitional terms that do not permit the inclusion additional components other than the components recited in the claims. The term “consisting essentially of” should be interpreted to be partially closed and allowing the inclusion only of additional components that do not fundamentally alter the nature of the claimed subject matter.

All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

All references, including publications, patent applications, and patents, cited herein are hereby incorporated by reference to the same extent as if each reference were individually and specifically indicated to be incorporated by reference and were set forth in its entirety herein.

Preferred aspects of this invention are described herein, including the best mode known to the inventors for carrying out the invention. Variations of those preferred aspects may become apparent to those of ordinary skill in the art upon reading the foregoing description. The inventors expect a person having ordinary skill in the art to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

### EXAMPLES

### Example 1

### Significance

Lymphedema is a debilitating condition in which the patient's upper or lower extremity experiences progressive fluid retention. Primary lymphedema is rare (1/100,00 children) while secondary lymphedema affects over 5 million individuals in the United States alone and is most often associated with lymph node surgery performed for cancer patients. In these patients despite a regimen of compression, patients experience progression to chronic lymphedema in which non-compressible fibrosis and adipose tissue are deposited within the affected extremity. In this study, we examine a strategy to mitigate fibrosis through delivery of a pro-adipogenic agent, rosiglitazone. We further determine that rosiglitazone also reduces adipose tissue deposition despite its role as a pro-adipogenic agent. These findings pave the way for pharmacologic inhibition of fibroadipose tissue deposition in lymphedema and provide a framework for the delivery of adjuvant therapy in conjunction with traditional compression therapy.

### Overview

Primary lymphedema is caused by alterations in genes responsible for the development of the lymphatic system, resulting in a lymphatic system that does not drain fluid properly. Secondary lymphedema occurs in up to 20% of patients after lymphadenectomy performed for the surgical management of tumors involving the breast, prostate, uterus, and skin. Patients develop progressive edema of the affected extremity due to retention of protein-rich lymphatic fluid. Compression therapy is cumbersome, and patients are only intermittently adherent to therapy. As a result, patients progress to chronic lymphedema in which non-compressible fibrosis and adipose tissue are deposited. The presence of fibrosis led to our hypothesis that rosiglitazone, a peroxisome proliferator-activated receptor gamma (PPARg) agonist which inhibits fibrosis, would reduce fibrosis in a mouse model of secondary lymphedema after hindlimb lymphadenectomy. We verified that rosiglitazone reverses the pro-fibrotic transcriptional effects of transforming growth factor-beta 1 (TGFβ1) in vitro. Rosiglitazone reduced fibrosis in the hindlimb after lymphadenectomy.

Our findings verified that rosiglitazone re-establishes the adipogenic features of TGFβ1-treated mesenchymal cells in vitro. Despite this, rosiglitazone led to a reduction in adipose tissue deposition. Flow cytometry demonstrated a reduction in the total presence of PDGFRα, a mesenchymal cell population which contribute to fibrosis and adipogenesis. While rosiglitazone increased the proportion of PDGFRα+ adipocytes (LipidTOX+) in the hindlimb, it reduced the absolute number of these cells. Furthermore, while lymphadenectomy led to increased size of PDGFRα+ adipocytes, rosiglitazone reversed this effect. Our findings provide a novel framework for treating secondary lymphedema as a condition of fibrosis and adipose tissue deposition the latter of which, paradoxically, can be prevented with a pro-adipogenic agent. They also provide new insight into our understanding of how these agents may be used to reduce adipose tissue deposition which occurs in a post-inflammatory niche.

### Introduction

Secondary lymphedema is a morbid condition, characterized by progressive limb swelling due to impaired drainage of lymphatic fluid. Patients develop retention of protein-rich lymphatic fluid which progresses to chronic lymphedema, characterized by limb hypertrophy caused by irreversible fibroadipose tissue deposition [Ref. 1]. As a result, patients experience limb heaviness, pain, open wounds, and disability, all of which severely impact quality-of-life for cancer patients [Ref. 2]. Overall, patients report reduced physical, functional, social, and emotional well-being [Ref. 3]. In the United States, this condition affects over 5 million individuals who have undergone removal of lymph nodes in the affected extremity for cancer surgery [Ref. 1].

Strategies to manage secondary lymphedema are inadequate. Non-surgical approaches such as sleeve, pneumatic pump compression, or manual lymphatic drainage demand 40+ hrs./wk of patient commitment and cause pain [Ref. 4]; even short periods of non-adherence lead to relapse [Ref. 5]. Surgical procedures to address secondary lymphedema [Ref. 6] have focused on reconstituting lymphatic drainage but are not curative and require continued long-term compression. Therefore, despite attempts to eliminate lymphatic fluid retention, almost all patients progress to some degree of chronic lymphedema.

The cellular mediators responsible for the deposition of fibroadipose tissue are poorly understood and may serve as a therapeutic target. Previous studies have implicated mesenchymal cells marked by expression of platelet-derived growth factor receptor-alpha (PDGFRα), as a cellular mediator of adipogenesis and fibrosis in alternate contexts. Therefore, we hypothesized that PDGFRα+ cells contribute to fibroadipose tissue deposition in secondary lymphedema. We further hypothesized that a strategy to induce adipogenic differentiation through delivery of rosiglitazone, a peroxisome proliferator-activated receptor gamma (PPARg) agonist, would paradoxically reduce fibroadipose tissue deposition in secondary lymphedema.

### Results

### Fibroadipose Tissue Deposition is Present in Patients with Chronic Secondary Lymphedema

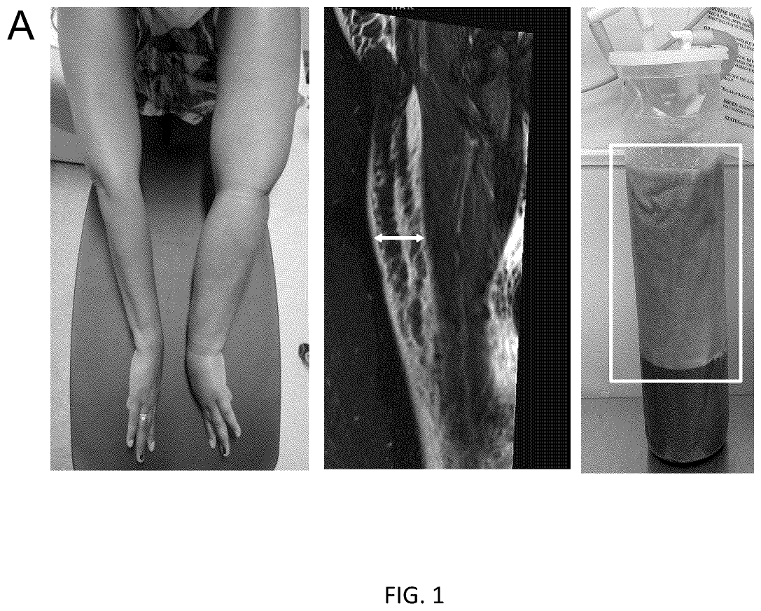

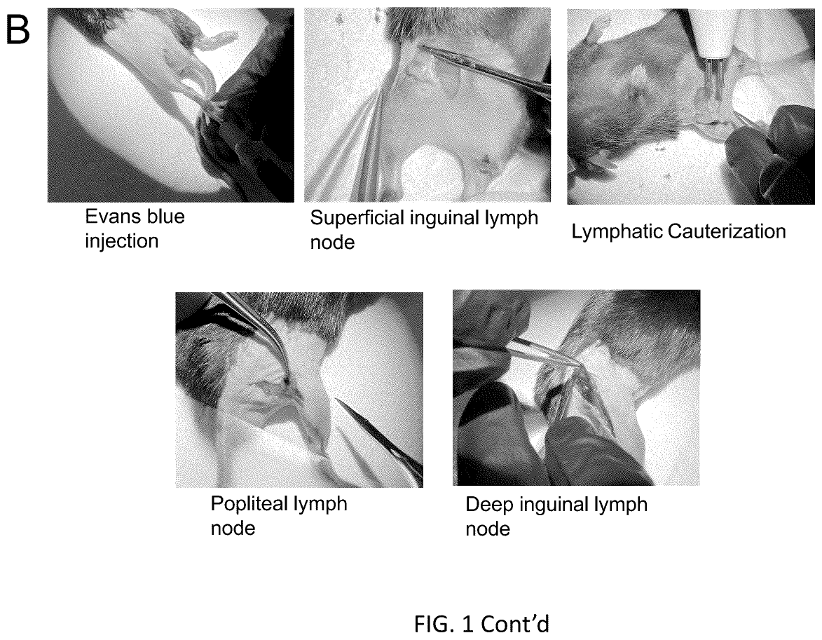

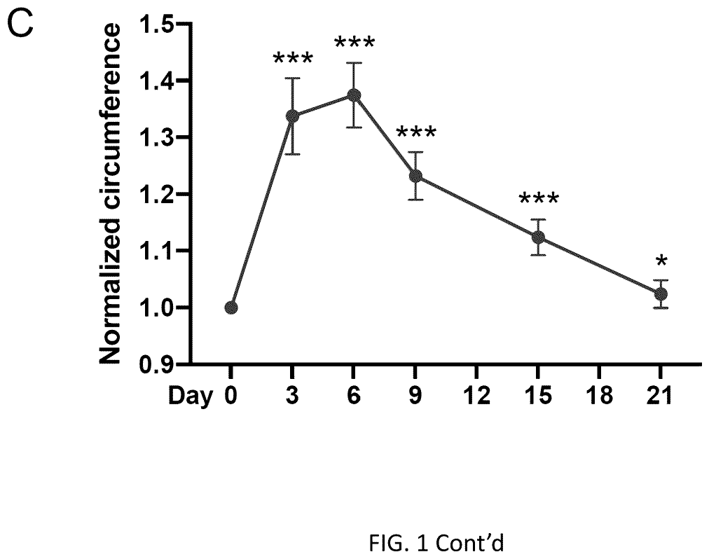

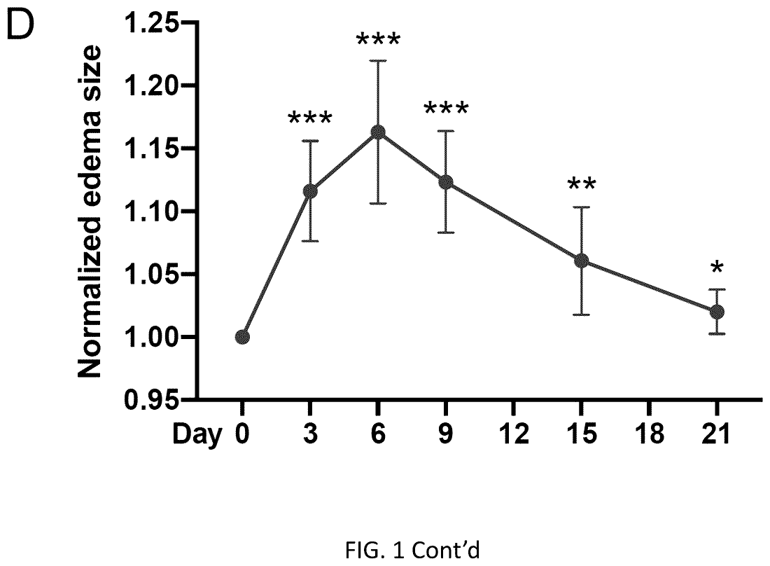

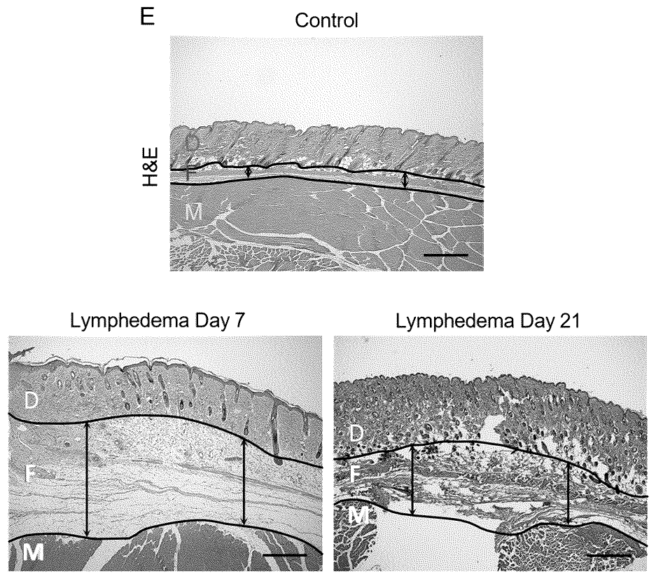

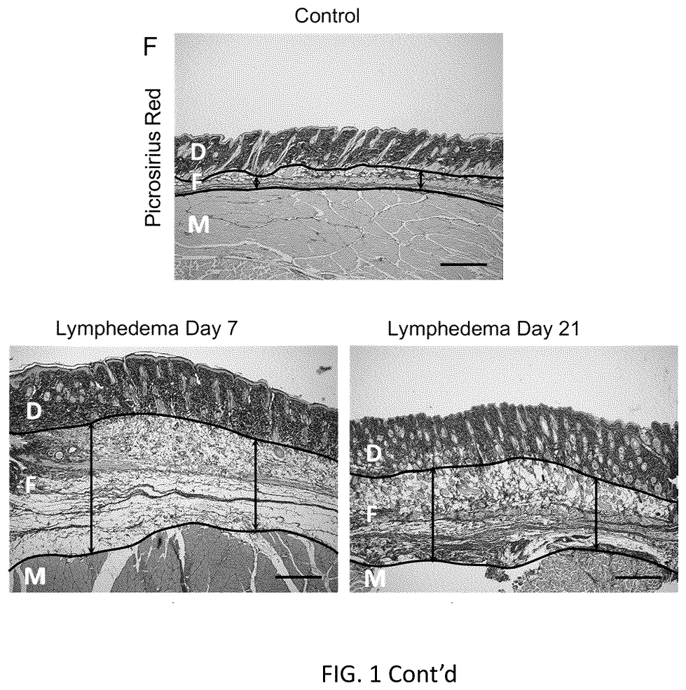

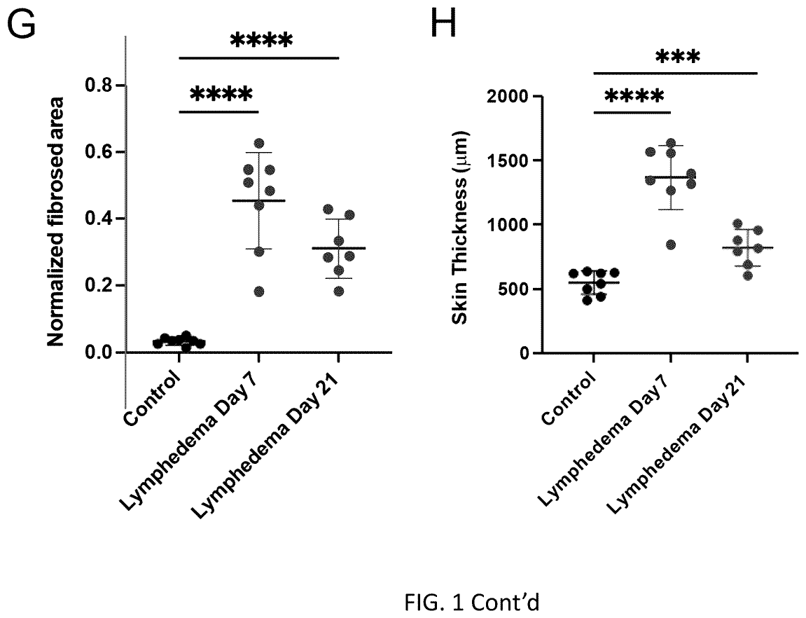

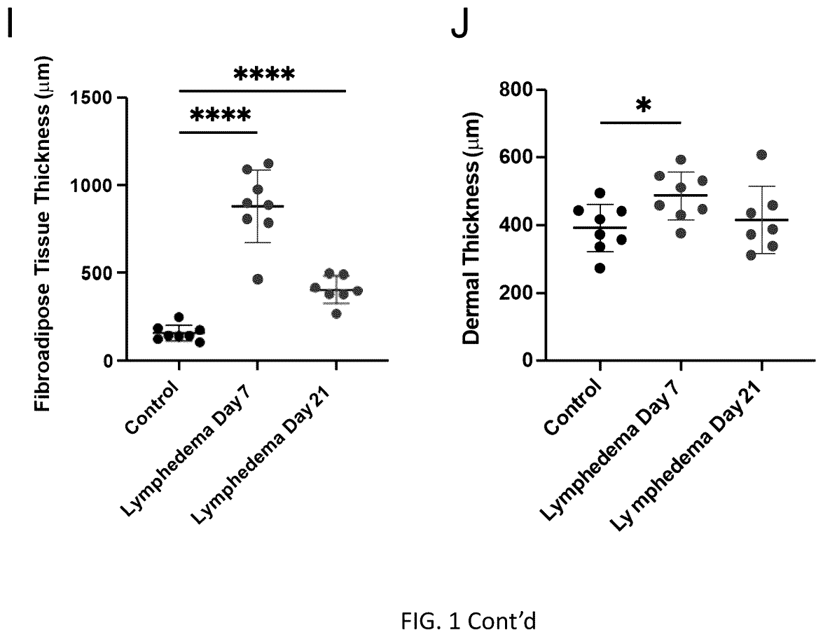

Using magnetic resonance imaging (MRI) in a patient with chronic secondary lymphedema of the upper extremity (FIG. 1 Panel A), we noted the presence of excess subcutaneous fibroadipose tissue based on T1-weighted MRI (FIG. 1 Panel A). Liposuction of the affected extremity verified the presence of excess adipose tissue which was directly removed (FIG. 1 Panel A).

### A Mouse Model of Secondary Lymphedema which Recapitulates Fibrosis and Adipose Tissue Deposition

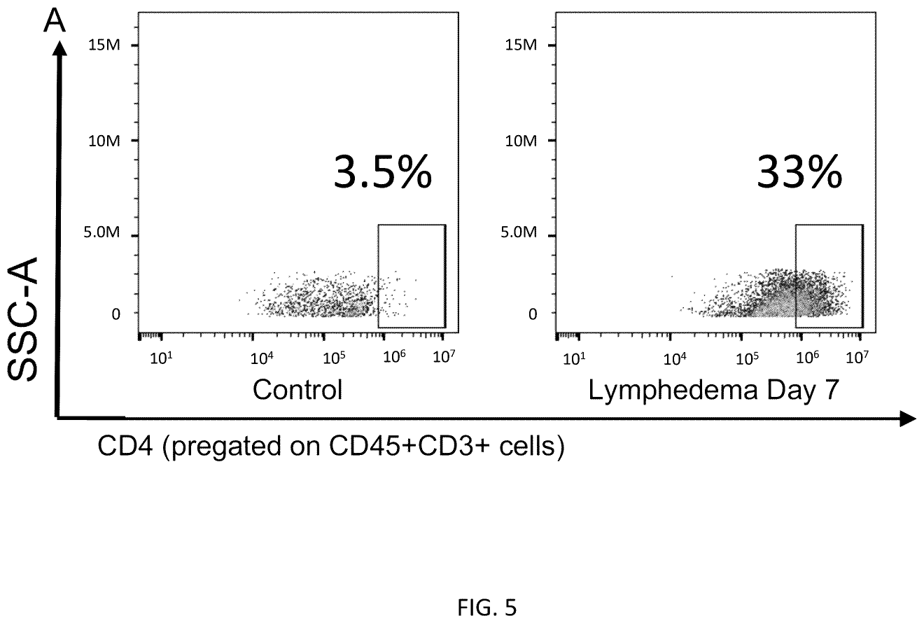

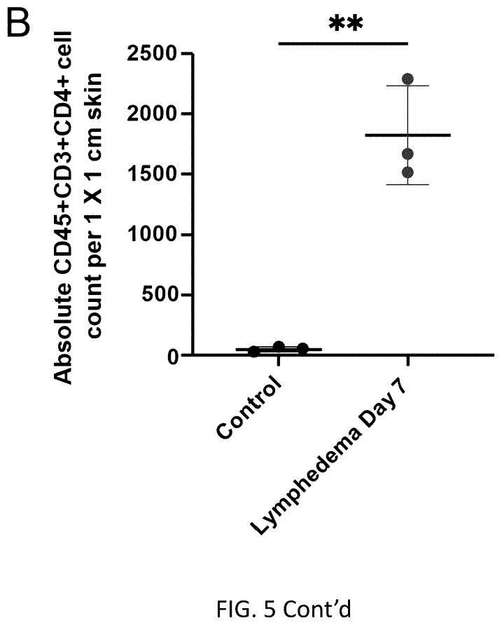

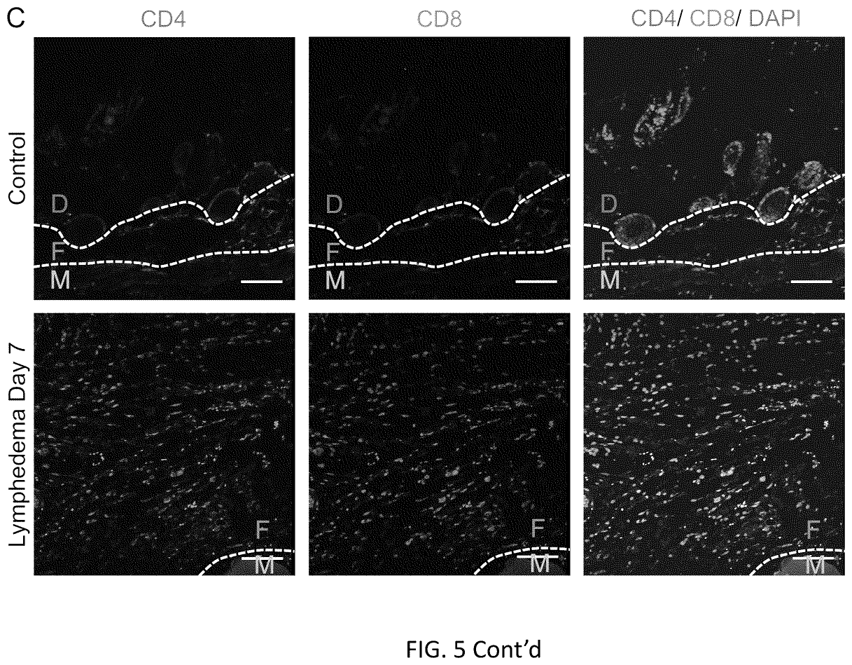

Hindlimb lymphadenectomy consisting of surgical excision of the hindlimb popliteal, superficial inguinal, and deep inguinal lymph nodes, and femoral lymphatic cauterization was performed (FIG. 1 Panel B) as previously described. Limb circumference, normalized to the uninjured hindlimb increased by nearly 40% within the first week after surgery (1.37 v. 1, p<0.001) (FIG. 1 Panel C). Similar findings were noted based on normalized edema (1.16 v. 1, p<0.001) (FIG. 1 Panel D). We noted a gradual improvement in both circumference and edema size based on gross measurements, although a significant increase persisted even after 3 weeks (circumference: 1.02 v. 1, p=0.044, edema size: 1.02 v. 1, p=0.024). We noted a marked increase in the presence of CD4+ T-cells of the affected hindlimb when compared with the control hindlimb based on flow cytometry (1.8×10² v. 0.5×10², p=0.0017) (FIG. 5 Panel A, Panel B) and immunohistochemistry (FIG. 5 Panel C).

Histologic evaluation was performed to further characterize changes in fibrosis and skin thickness after lymphadenectomy. Based on picrosirius red staining (FIG. 1 Panel F), we noted increased fibrosis in the affected hindlimb at both 1 and 3 weeks after lymphadenectomy (1 week: 0.454 v. 0.033, p<0.0001; 3 week: 0.311 v. 0.033, p<0.0001) (FIG. 1 Panel G). Based on H&E staining, we noted increased thickness of the skin (marked D and F) and of the subdermal layer alone (marked F) at both 1 and 3 weeks after lymphadenectomy (1 week: skin: 1365.04 μm v. 548.88 μm, p<0.0001, subdermal layer: 878.36 μm v. 157.77 μm, p<0.0001; 3 week: skin:

819.10 μm v. 548.88 μm, p<zzz, subdermal layer: 403.73 μm v. 175.77 μm, p<0.0001) (FIG. 1 Panel E, Panel H, Panel I). We also noted an increase in the dermal thickness (marked D) at 1 week after lymphadenectomy, which later improved after 3 weeks (1 week: 486.67 μm v. 391.11 μm, p=0.0175; 3 week: 415.37 μm v. 391.11 μm, p=0.5917) (FIG. 1 Panel J).

### Augmented PPARg Signaling Reduces Fibrosis and Adipose Tissue Deposition

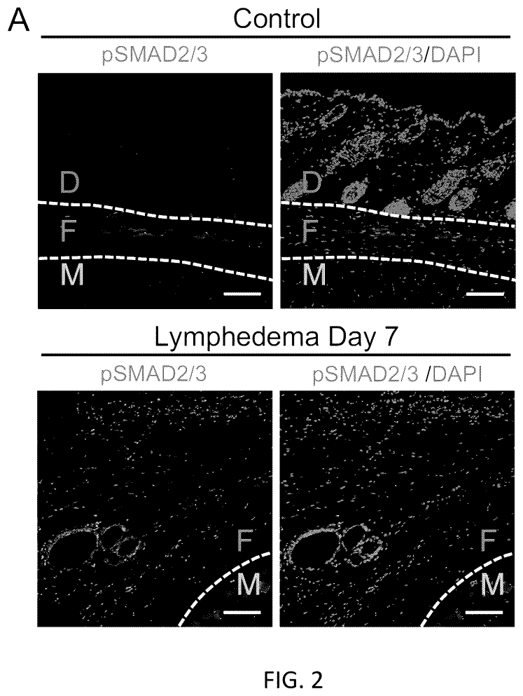

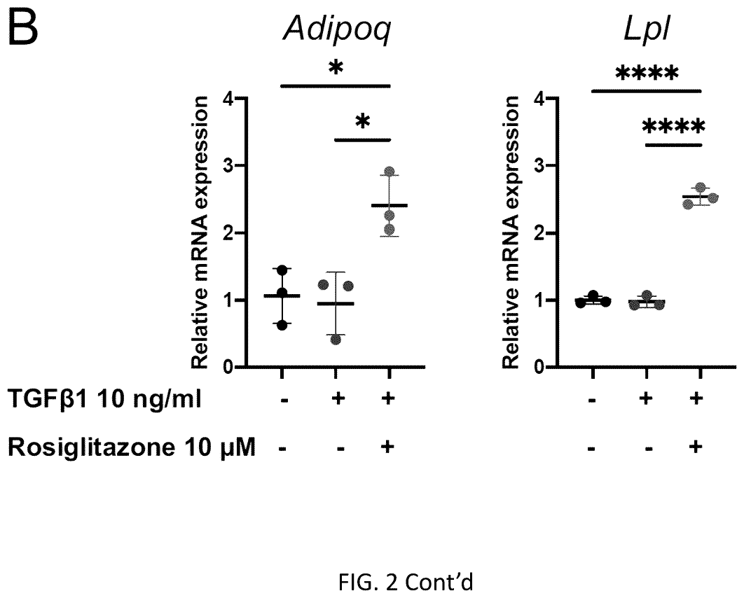

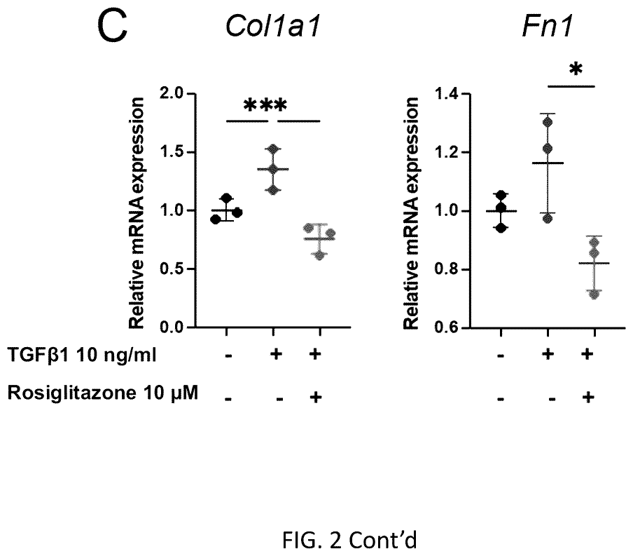

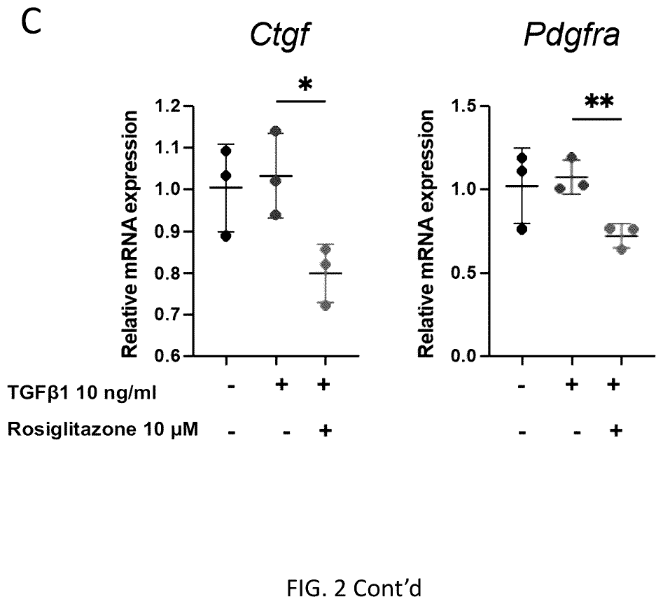

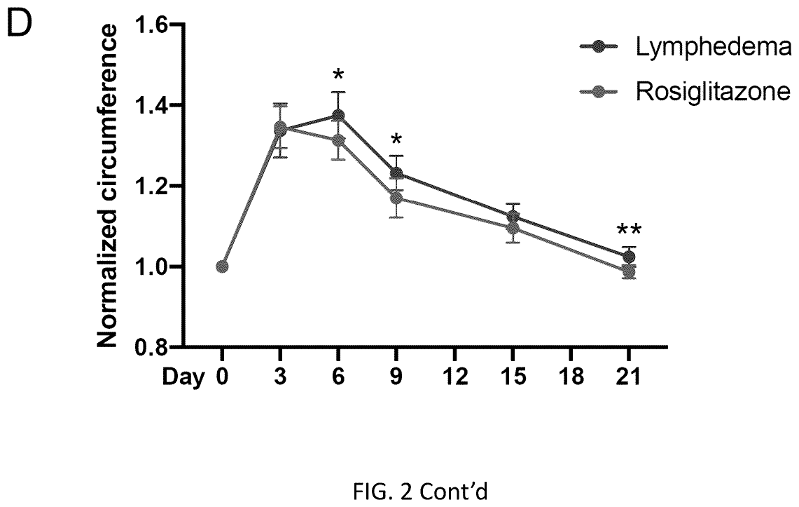

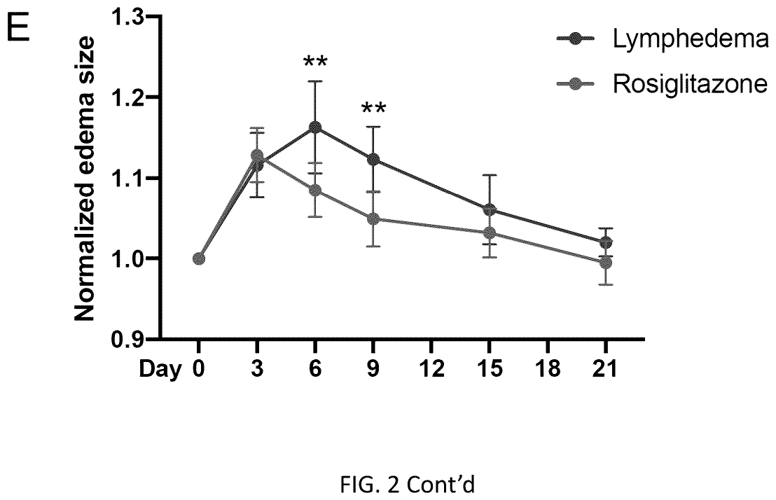

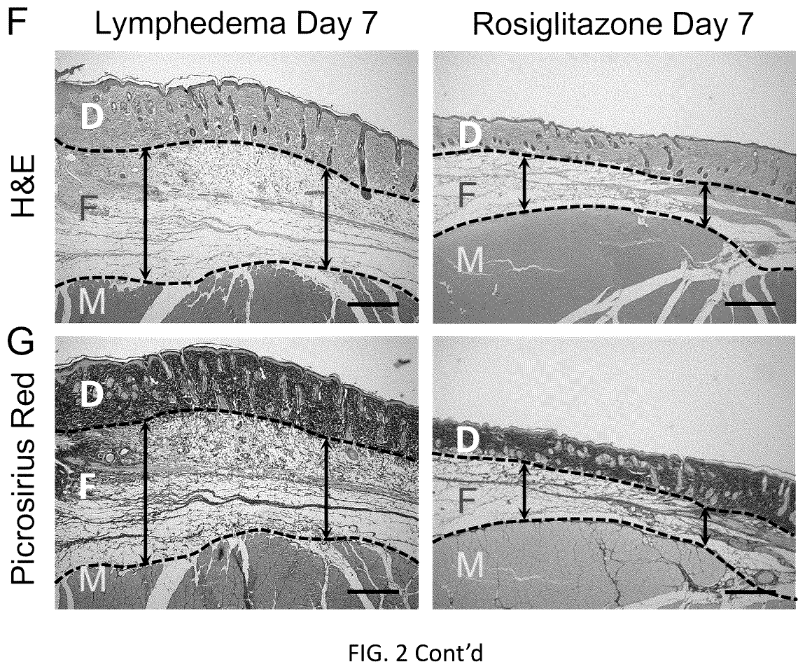

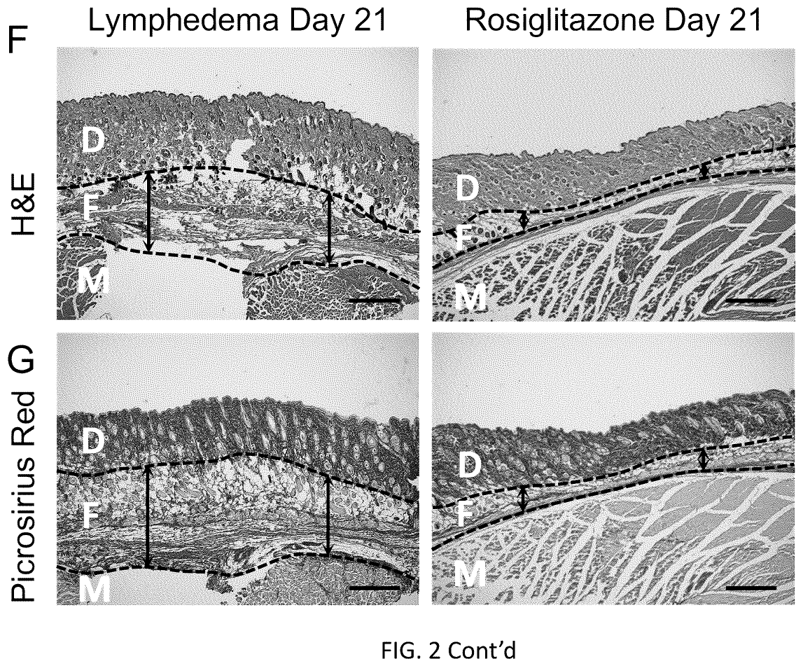

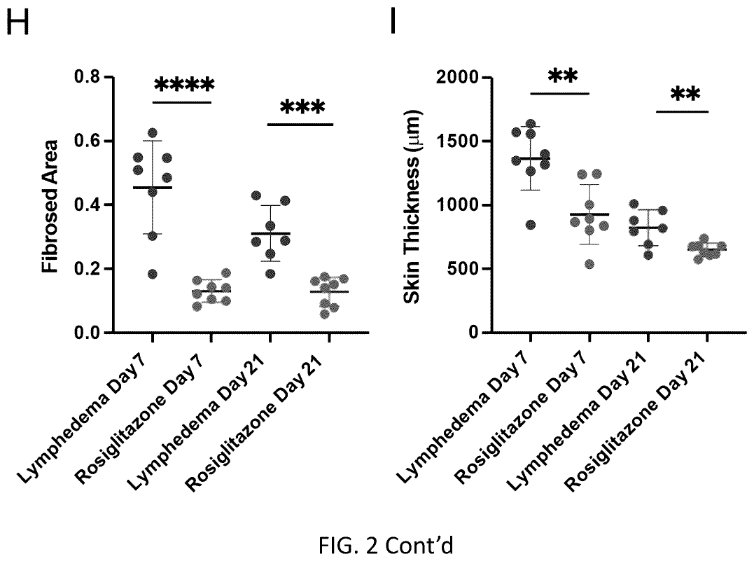

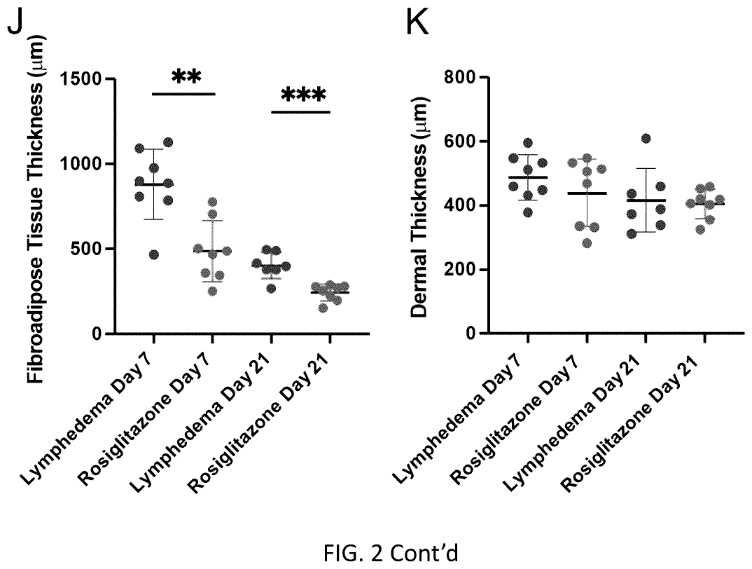

Next, we sought to reverse the fibrosis observed in secondary lymphedema through augmented peroxisome proliferator-activated receptor gamma (PPARg) activity. Previous studies have established that PPARg agonists reverse the effects of pro-fibrotic transforming growth factor-beta (TGFβ) ligands. First, we examined the presence of increased TGFβ signaling in the hindlimb after lymphadenectomy; immunostaining for phospho-Smad 2/3 verified increased signaling after lymphadenectomy (FIG. 2 Panel A). Based on this confirmation, we designed an in vitro experiment in which adipose-derived mesenchymal stem cells (AdMSCs) were treated with TGFβ1 ligand and rosiglitazone, with subsequent evaluation of adipogenic and fibrogenic gene expression; while TGFβ1 reduced adipogenic gene expression among AdMSCs, rosiglitazone reversed this anti-adipogenic effect (Adipoq: 0.95 v. 2.40, p=0.0177; Lpl: 2.53 v. 0.97, p<0.0001) (FIG. 2 Panel B). Furthermore, rosiglitazone reduced fibrogenic gene expression which had been induced by TGFβ1 (Col1a1: 1.35 v. 0.76, p=0.0091; Fn1: 1.16 v. 0.82, p=0.038, Ctgf: 1.03 v. 0.80, p=0.0306, Pdgfra: 1.07 v. 0.72, p=0.0083) (FIG. 2 Panel C).

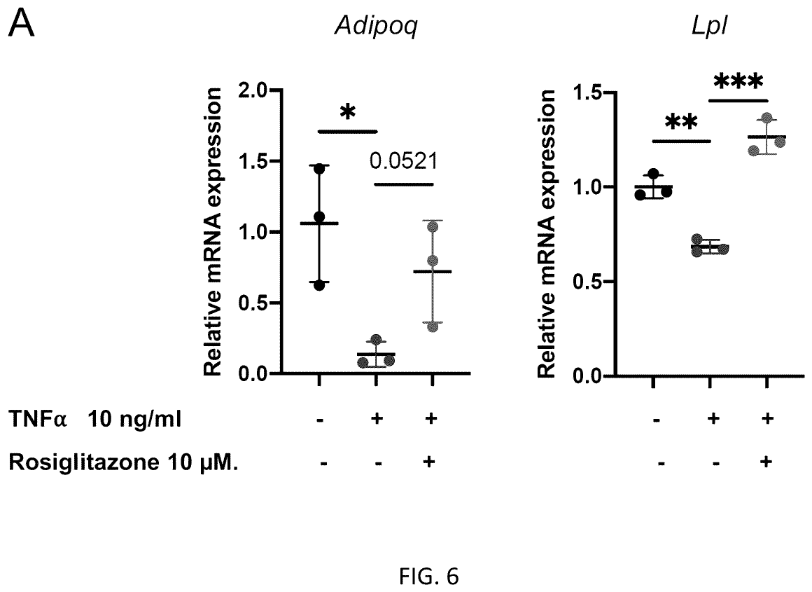

Similarly, rosiglitazone rescued adipogenic gene expression which was reduced among TNFα-treated AdMSCs (FIG. 6). These findings provided support for in vivo delivery of rosiglitazone to mice which had undergone lymphadenectomy. We noted modest initial reductions in limb circumference and edema after 1 week (circumference: 1.31 v. 1.37, p=0.0354, edema size: 1.08 v. 1.16, p=0.0048) (FIG. 2 Panel D, Panel E). Furthermore, picrosirius red staining and quantification verified the desired reduction in fibrosis (1 week: 0.130 v. 0.454, p<0.0001; 3 week: 0.128 v. 0.311, p<0.001) (FIG. 2 Panel G, Panel H). Upon detailed histologic evaluation however, we noted an unexpected reduction in subdermal adipose tissue and overall skin thickness (1 week: skin: 924.66 μm v. 1365.04 μm, p=0.0027, subdermal layer: 486.12 μm v. 878.36 μm, p=0.0012; 3 week: skin: 646.12 μm v. 819.10 μm, p=0.0068, subdermal layer: 242.36 μm v. 403.73 μm, p=0.0003) (FIG. 2 Panel F, Panel I, Panel J). Interestingly, we did not identify a reduction in the dermal thickness (1 week: 438.54 v. 486.67, p=0.3034; 3 week: 403.76 v. 415.37, p=0.7702) (FIG. 2 Panel K).

### Increased Presence of Adipogenic PDGFRα· Cells after Lymphadenectomy is Reduced by Rosiglitazone

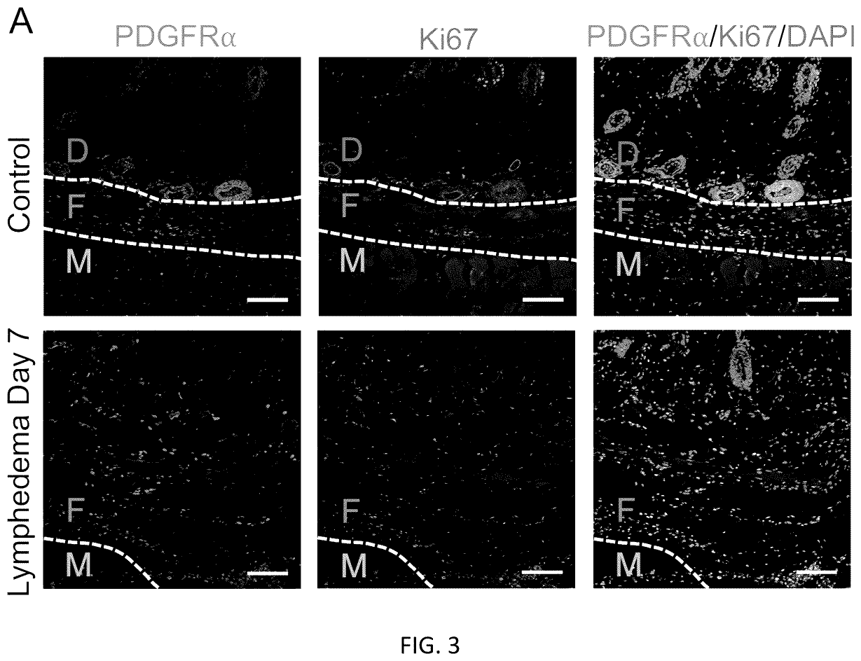

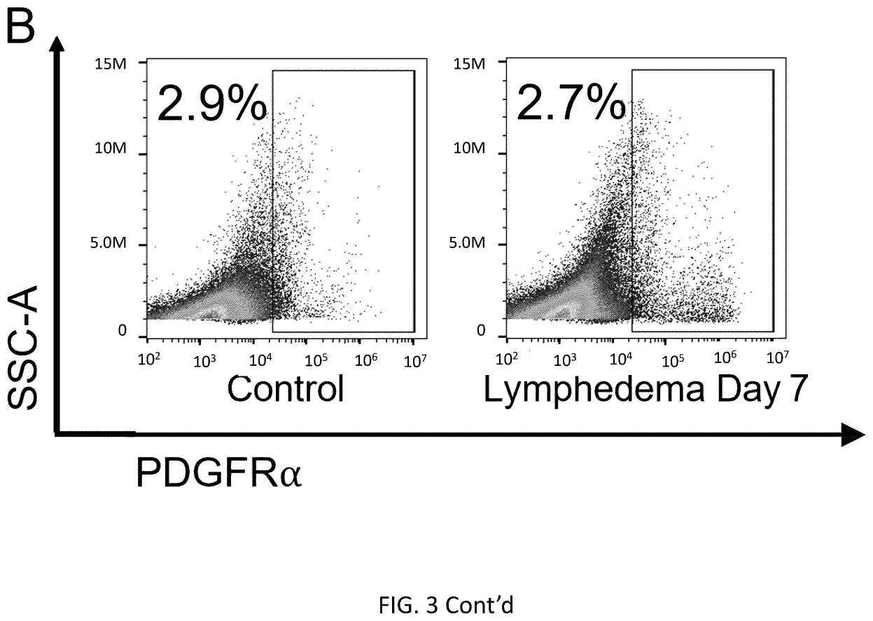

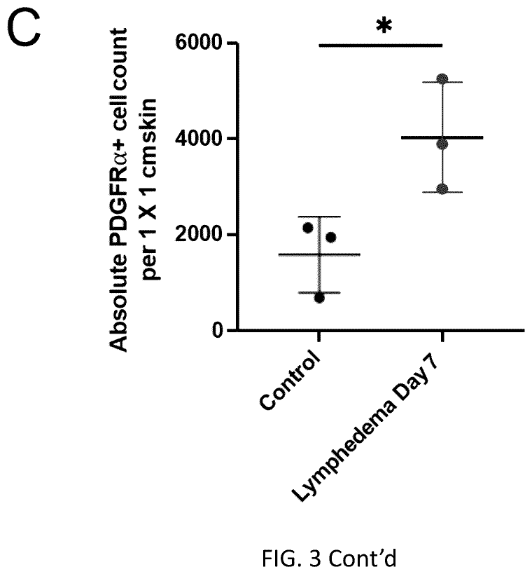

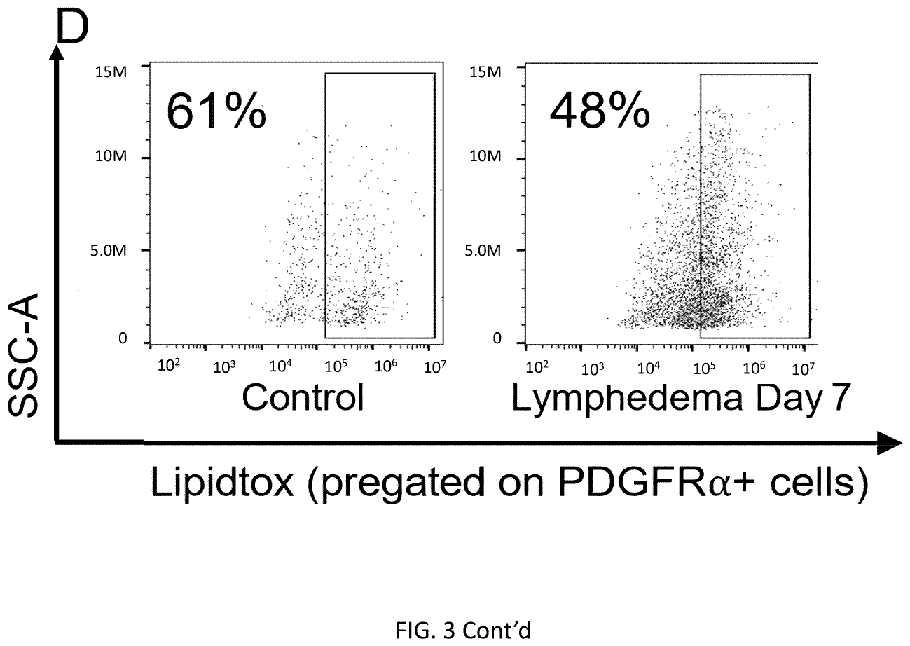

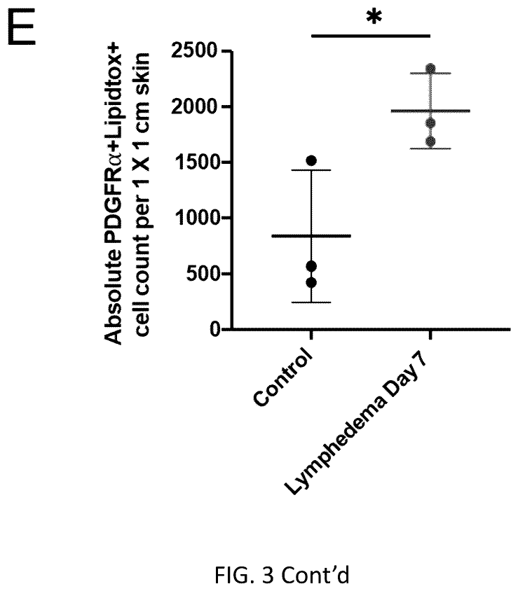

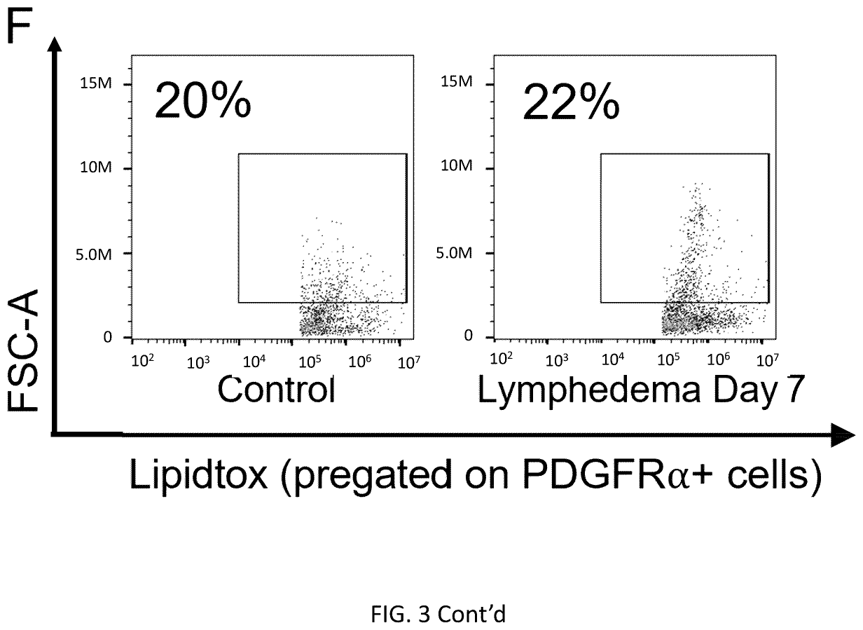

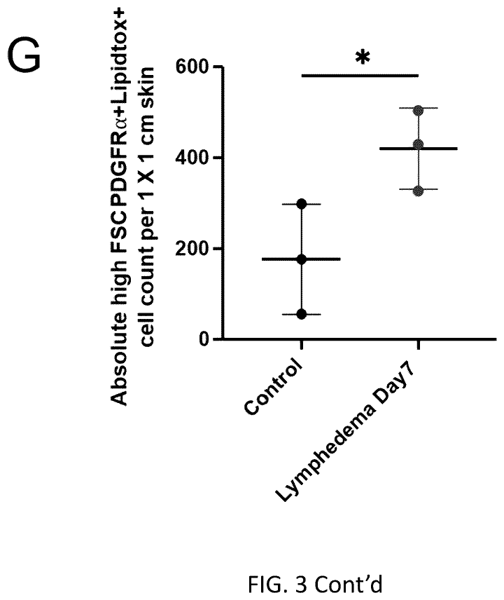

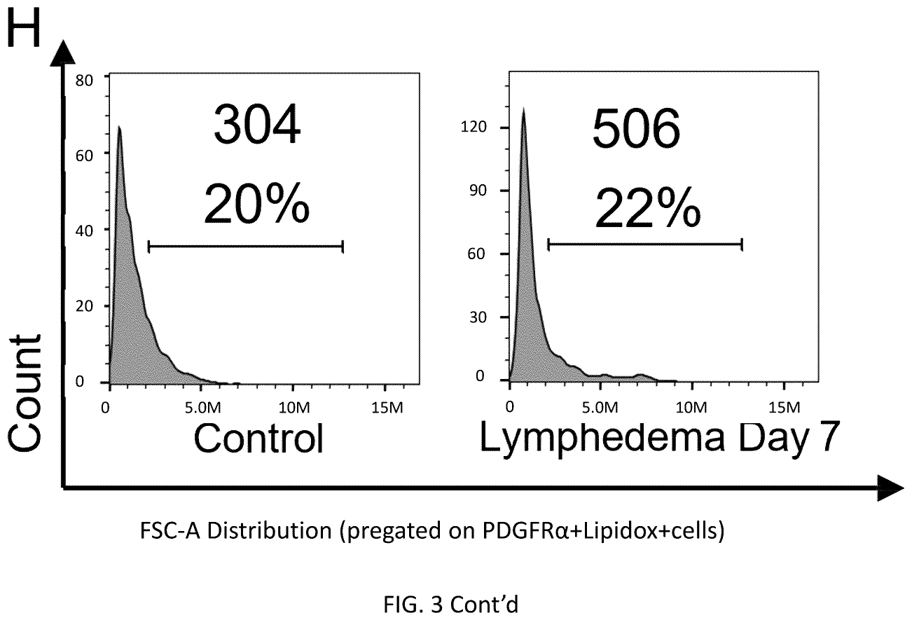

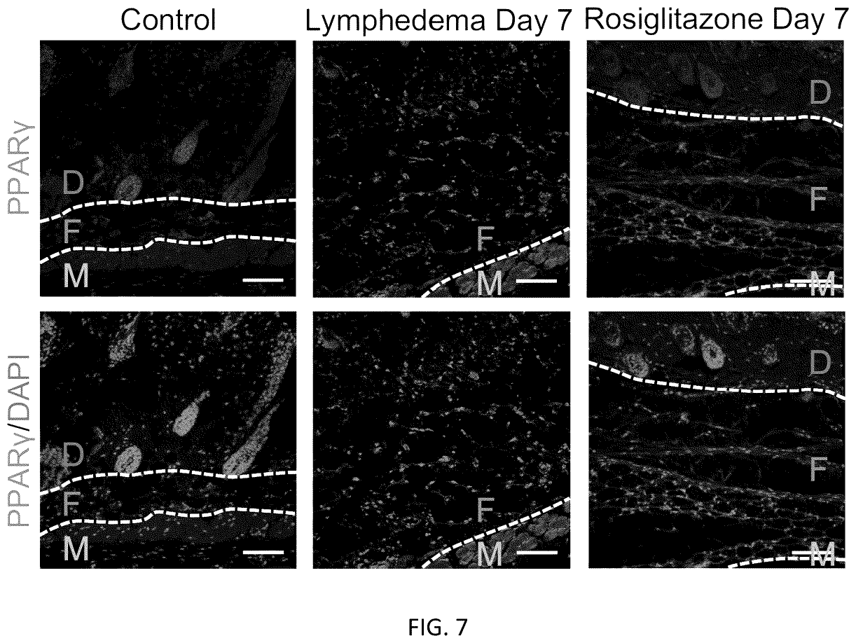

Given the unexpected finding of reduced subdermal adipose tissue in rosiglitazone-treated mice, we first verified that PPARg levels were not reduced as a result of rosiglitazone treatment (FIG. 7). Next, we sought to examine how rosiglitazone modifies PDGFRα+ cells, which have been previously shown to contribute to both fibrosis and adipogenesis. The hindlimbs of mice euthanized 1 week after lymphadenectomy were evaluated for the presence of PDGFRα+ cells. Immunostaining verified a visible increase in the presence of PDGFRα+ cells (FIG. 3 Panel A). Co-staining with Ki67 verified that these cell populations exhibited proliferation within the site (FIG. 3 Panel A). Flow cytometry performed over a standard 1 cm×1 cm area of skin similarly demonstrated a significant increase in the absolute count of PDGFRα+ cells after lymphadenectomy relative to the uninjured hindlimb (4.0×10³ v. 1.6×10³, p=0.0392) (FIG. 3 Panel B, Panel C). Upon staining with LipidTOX, we noted a significantly increased presence of PDGFRα+LipidTOX+ cells indicative of their adipogenic features (2.8×10³ v. 1.1×10³, p=0.0092) (FIG. 3 Panel D, Panel E). Forward scatter assessed by flow cytometry demonstrated increased size of PDGFRα+LipidTOX+ cells in the setting of lymphadenectomy relative to the unaffected hindlimb (FSC^{high}: 5.5×10² v. 2.5×10², p=0.0303) (FIG. 3 Panel F, Panel G).

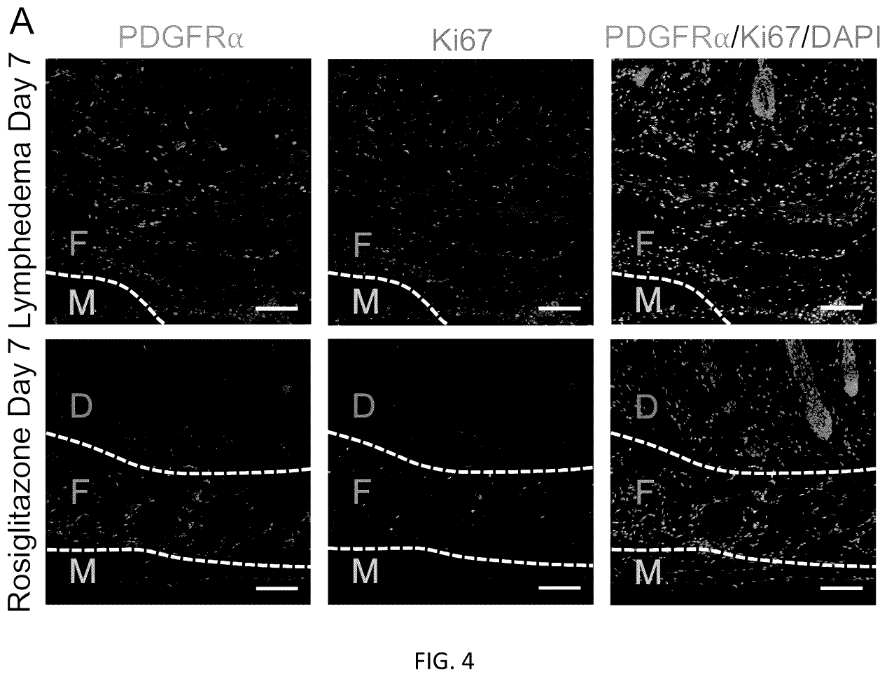

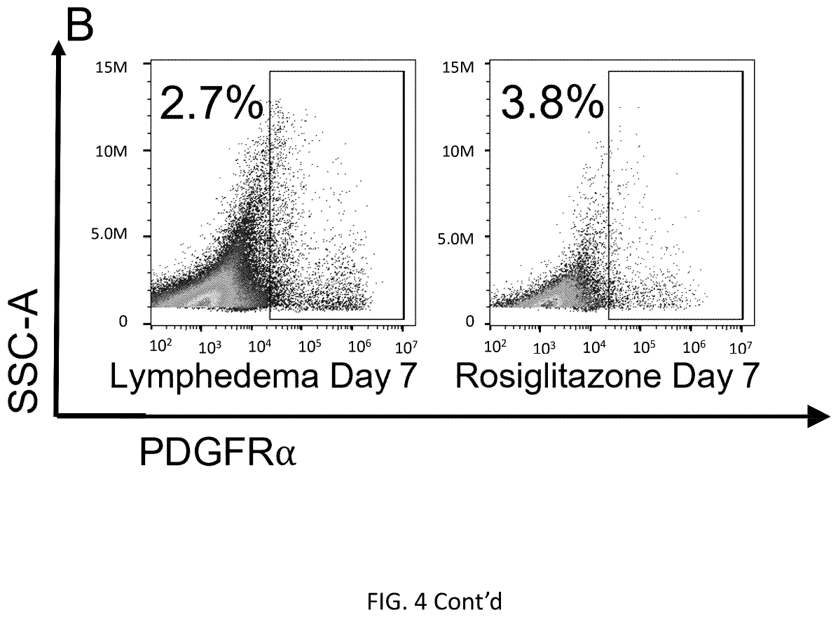

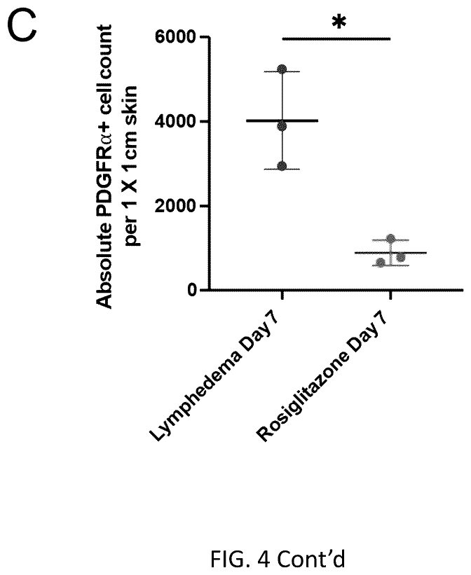

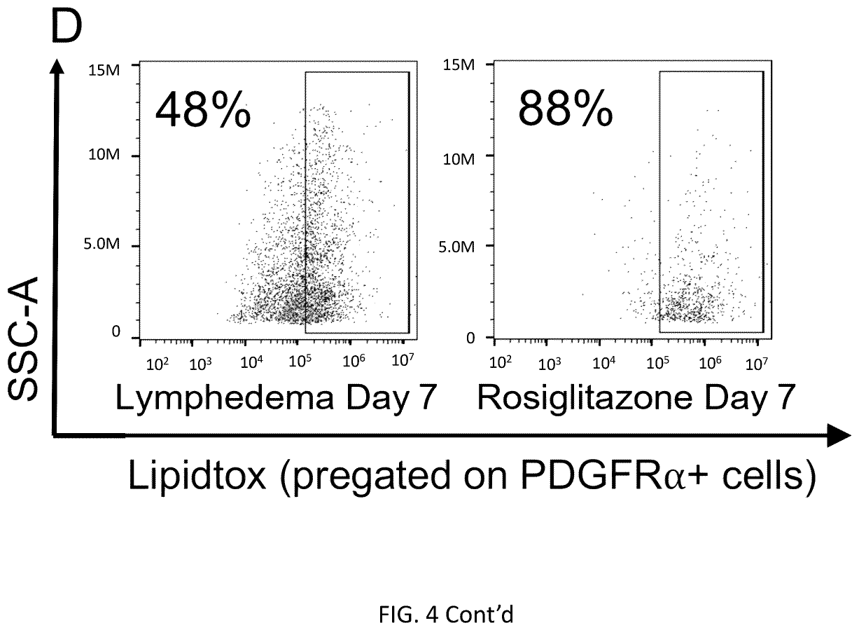

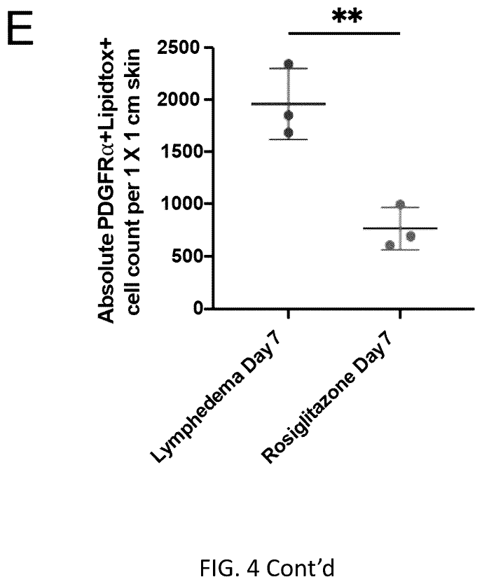

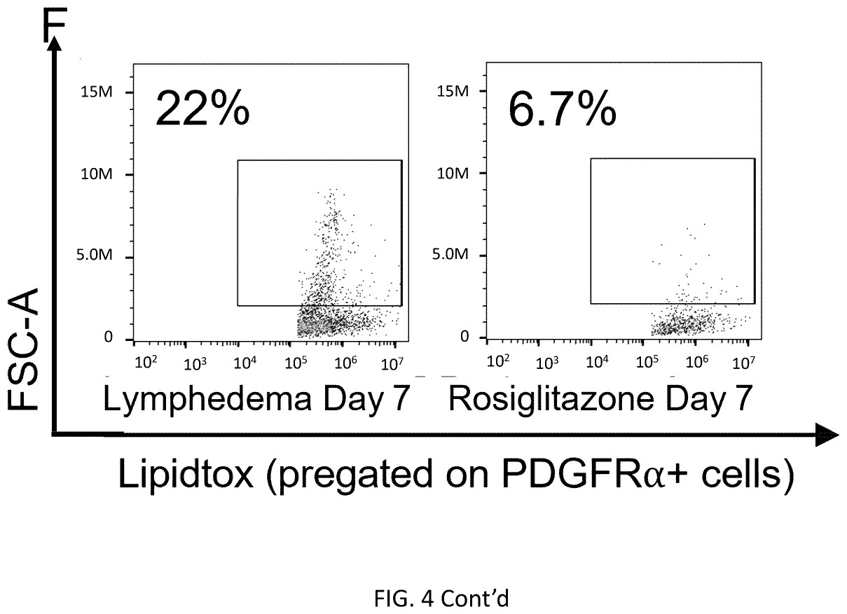

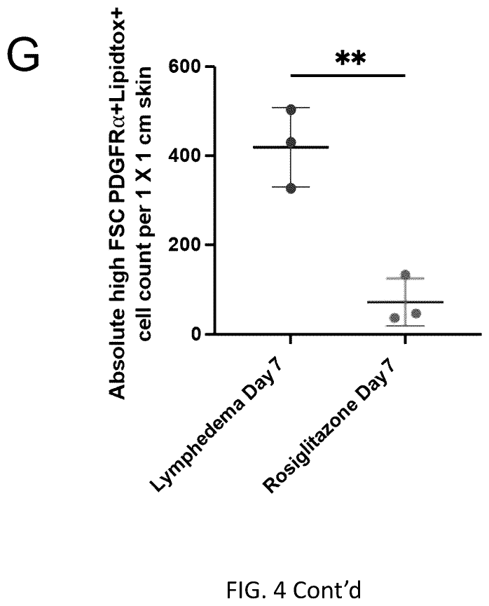

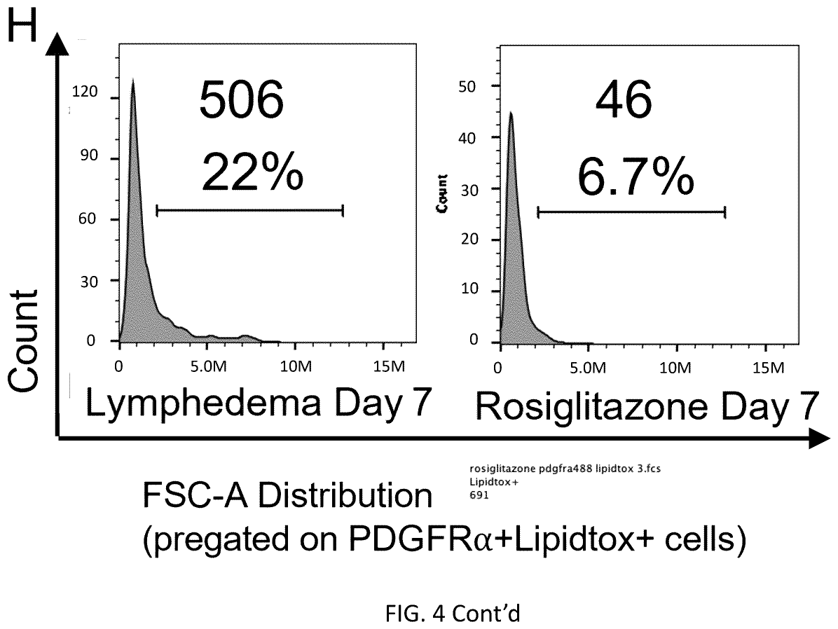

The hindlimbs of mice treated with rosiglitazone after lymphadenectomy were compared with those which did not received rosiglitazone. Immunostaining verified a reduction in the presence of PDGFRα+ cells (FIG. 4 Panel A) with rosiglitazone treatment; there was a corresponding reduction in PDGFRα+Ki67+ cells (FIG. 4 Panel A). Flow cytometry quantified a significant and substantial reduction in PDGFRα+ cells within a standardized 1 cm×1 cm area of skin (0.9×10³ v. 4.0×10³, p=0.0104) (FIG. 4 Panel B, Panel C). Flow cytometry for PDGFRα+LipidTOX+ cells demonstrated an increase among rosiglitazone-treated samples as a proportion of total PDGFRα-cells (86% v. 53%, p=0.0788). However, the absolute number of PDGFRα+LipidTOX+ cells was reduced in rosiglitazone-treated mice (862 v. 2842, p=0.0004) (FIG. 4 Panel E). Finally, using flow cytometry, we were able to quantify the change in the size of PDGFRα+LipidTOX+ cells, showing a reduction with rosiglitazone treatment (FSC^{high}: 73 v. 553, p=0.0005) (FIG. 4 Panel F, Panel G).

### Discussion

Secondary lymphedema affects 5 million individuals in the United States alone [Ref. 1]—a number greater than the number of individuals living with multiple sclerosis, rheumatoid arthritis, and lupus combined, and approximating the number of individuals with Alzheimer's disease. Therefore, secondary lymphedema presents a substantial healthcare burden. In the United States, this condition typically affects cancer survivors who have undergone lymphadenectomy. The morbidity associated with secondary lymphedema is substantial, including limb hypertrophy, soreness, heaviness, pain, and infection, all leading a significantly reduced body image and quality of life [Ref. 1,7-9]. In fact, the morbidity of secondary lymphedema has led to the development of myriad clinical trials studying the efficacy of surgical treatment without lymphadenectomy (e.g., Z0011 breast cancer and MSLT melanoma trials) [Ref. 10-14]. However, despite these studies, lymphadenectomy remains a component of surgical management of cancers of the breast, skin, head/neck, prostate, and uterus, among others.

The treatment of secondary lymphedema has been traditionally limited to non-surgical management including compression, pneumatic pumps, and lymphatic massage. However, these strategies are cumbersome and require long-term adherence as they are unable to reverse the underlying pathology. Due to low long-term adherence with these therapies [Ref. 15], patients often progress to a state of chronic lymphedema characterized by fibroadipose tissue deposition. Based on the severity of this condition, liposuction has emerged as a surgical strategy to directly remove the fibroadipose tissue from affected limbs [Ref. 16-21]. Although this has demonstrated efficacy with both reducing limb hypertrophy, alleviating patient symptoms including heaviness and pain, and reducing infection risk [Ref. 22], surgery presents several limitations. First, surgery is not accessible to all patients, which is a major limitation given the number of patients affected by this condition.

Second, patients often require multiple liposuction procedures, thereby further impacting accessibility. Third, this procedure requires post-operative care including the management of potential wounds. Fourth, this procedure may not be covered by insurance policies for many patients. Finally, the surgical approach is reactive, and does not prevent the initial development of fibroadipose tissue. Together, these provide a strong need for non-surgical strategies which improve accessibility, obviate surgical recovery, are cost-effective, and prevent the initial formation of fibroadipose tissue. Such a strategy could be implemented in conjunction with traditional non-surgical compression.

Our results show that rosiglitazone, a pro-adipogenic agent which augments signaling through peroxisome proliferator-activated receptor gamma (PPARg), reduces fibrosis and fibroadipose tissue thickness in the hindlimb. These findings are consistent with previous studies indicating that rosiglitazone reduces fibrosis in other disease contexts including liver [Ref. 24], lung [Ref. 25], and dermal fibrosis [Ref. 26]. For example, rosiglitazone reduces expression of pro-fibrotic peptides including Ctgf, α-SMA, and Col1 by dermal fibroblasts in scleroderma [Ref. 26]. Our in vitro studies demonstrate that rosiglitazone indeed reduced the expression of pro-fibrotic genes by adipose-derived mesenchymal cells which were exposed to pro-fibrotic transforming growth factor-beta 1 (TGFβ1).

Our findings also verified the pro-adipogenic effect of rosiglitazone in the face of TGFβ1 or tumor necrosis factor-α (TNFα), both ligands which are present during the development and progression of secondary lymphedema. Unexpectedly, our histologic examination of the hindlimb demonstrated a reduction in the total amount of fibroadipose tissue in the subdermal layer with a reduction in the number of adipocytes based on histology. These findings led us to examine how rosiglitazone may impact PDGFRα+ cells—these cells have been implicated as cellular contributors to both fibrosis [Ref. 27] and to adipocyte formation. Using PDGFRα lineage-tracing mice Joshi et al showed that PDGFRα+ cells present in the mammary stroma contribute to mammary adipocytes [Ref. 28]. Scherer et al also showed that adipocytes undergo reversible de-differentiation into proliferative PDGFRα+ mesenchymal cells, which are then capable of undergoing re-differentiation into mature adipocytes [Ref. 29]. Finally, Shin et al showed that embryonic PDGFRα-cells contribute to mature adipocytes during development. In our experiments immunostaining and flow cytometry verified the increased presence of PDGFRα+ cells in the lymphedematous hindlimb relative to control. Moreover, these PDGFRα+ cells were proliferative and exhibited lipid accumulation based on staining with LipidTOX. However, rosiglitazone reduced the total number of both PDGFRα+ and LipidTOX+ PDGFRα+ cells, suggesting that its effect was to reduce proliferation of PDGFRα+ cells. This was further confirmed based on flow cytometry which showed that over 90% of PDGFRα+ cells in the rosiglitazone-treated samples were LipidTOX+; this finding in particular provides strong evidence that although higher proportion of PDGFRα+ cells underwent adipogenic differentiation with rosiglitazone treatment, rosiglitazone reduced the overall presence of these cells.

Notably, lymphedema led to an increase in the mean size of PDGFRα+ adipocytes. However, rosiglitazone treatment appeared to reduce adipocyte size based on forward scatter using flow cytometry. This finding is consistent with the reported effect of rosiglitazone on adipocytes in other contexts where it has been shown to induce more compact adipocytes with smaller lipid inclusions [Ref. 30]. Therefore, it appears that the effect of rosiglitazone is multi-modal, through a reduction in fibrosis, a reduction in total PDGFRα+ which are capable of adipogenic differentiation, and a reduction in the size of PDGFRα+ adipocytes.

We recognize several limitations of this study. First, while the model of secondary lymphedema presented here is representative of the human condition both based on the surgical lymphadenectomy and the development of fibroadipose tissue deposition, the timeline of this development is accelerated in the mouse model. Although other models such as the tail model have gained in popularity, the circumferential skin incision is not representative of the extensive lymphadenectomy performed in patients. In addition, the tail model also experiences gradual resolution of the underlying lymphedema over the course of several weeks. Importantly, our model does recreate the increased CD4+ T-cell infiltration observed in the tail lymphedema model.

Secondly, the question of when to administer rosiglitazone therapy is critical. Based on our clinical experience patients are often intermittently adherent to therapy; because of this, all patients progress to some degree of fibroadipose tissue deposition. As a result, this study paves the way for inquiries into the use of rosiglitazone as an adjunct therapy initiated simultaneously with non-surgical compression therapy. Third, rosiglitazone is associated with adverse effects including heart failure; however, these findings have been noted in patients with an underlying history of diabetes, for which rosiglitazone was originally indicated. Fourth, questions remain regarding the source of PDGFRα+ cells present in the lymphedema site—it remains unclear whether these cells are derived from local mesenchymal cells or from cells which traffic to the injury site. Improved understanding of the source of these cells may provide clues to additional therapeutic strategies.

The present Example presents a therapeutic agent which simultaneously addresses the fibrosis and adipose tissue deposition present in secondary lymphedema by modifying the fate and function of mesenchymal cells in the injury site. Based on these preclinical findings, we anticipate a clinical trial examining rosiglitazone therapy in patients with early-stage lymphedema who would benefit with prevention of progression to chronic lymphedema.

### Methods

### Animals

6-8-week old male C57BL/6J mice (Jackson Laboratories; Bar Harbor, ME, USA; weight: 23±2 g) were acclimatized for one week in the Brigham and Women's Hospital (BWH) vivarium. The experiments were carried out according to the protocol approved by the Institutional Animal Care and Use Committee (IACUC) at BWH.

### Mouse Hind Limb Lymphedema Model

Evans blue (Sigma) (4% in PBS) was filtered and injected into mouse foot pad to stain lymph nodes in hind limb. Mouse hind limb secondary lymphedema was induced by surgical excision of the ipsilateral superficial inguinal, popliteal, deep inguinal lymph nodes, and the femoral lymphatic vessel. During surgical procedures, electrocauterization were used to prevent bleeding.

### Animal Experimental Setup and Rosiglitazone Treatment

A total of 32 mice underwent lymphedema surgery were randomly assigned into two groups, lymphedema group and rosiglitazone group (n=16). Rosiglitazone (Combi-Blocks, San Diego, USA) was dissolved in DMSO at the concentration of 25 mg/ml, and was diluted in corn oil to the final concentration of 2.5 mg/ml before injection. Mice in rosiglitazone group were intraperitoneally injected with rosiglitazone (10 mg/kg) twice a week from the day of surgery (day 0, 3, 7, 10, 14, 17). Mice in lymphedema group were injected with corn oil only at same time point. 8 mice in lymphedema group and rosiglitazone group were sacrificed on day 7 (n=8 per group), the rest of them were sacrificed on day 21 (n=8 per group). Another 18 mice were randomly divided into three groups, normal control group, lymphedema group, and rosiglitazone group, skin cells from these mice were isolated and used for flow cytometry analysis at day 7 (n=3 per group per staining).

### Tissue Procurement

Sacrifice was performed via CO₂ asphyxiation. Skin and muscle of hind limb were harvested, fixed in 10% formalin for 48 h followed by 70% ethanol for histological assessment. Formalin-fixed tissues were then dehydrated, embedded in paraffin, and sectioned with 5-micron-thickness.

### Histology

Sections were stained either with Hematoxylin and Eosin (H&E) or Picrosirius Red according to standard protocol, and used to assess the overall skin thickness, dermal thickness, fibroadipose tissue thickness, and fibrosis, respectively. Quantitative histological analysis was performed using samples from day 7 and 21.

Images were captured by using Olympus BX53 (UCMAD3, T7, Japan) and all-in-one Keyence (Itasca, IL, USA) microscopes and assessed by ImageJ (version 1.52a; Media Cybernetics, Rockville, MD, USA) by two independent researchers under blinded conditions. Fibrosis was assessed with the image thresholding plugin in ImageJ software, normalization is based on the length of the section (v1.52a; Media Cybernetics, Rockville, MD, USA).

### Immunofluorescence Staining

Tissue sections were de-paraffinized, rehydrated and probed with antibodies for APC-PDGFRα (1:100), APC-PDGFRβ (1:100), FITC-CD4 (1:50), Alexa Fluor 647-CD8b (1:50), Ki67 (1:200), F4/80 (1:100), pSMAD2/3

(1:200) and PPAR-gamma (1:200). The sections were blocked with 10% goat serum, 1% Bovine serum albumin (BSA), and 0.3% Triton (Sigma) in PBS for one hour and incubated with the primary antibodies at 4° C. overnight. After thorough washing, the sections where incubated for one hour in the dark using the following secondary antibodies: Alexa Fluor Plus 488 goat anti-rabbit IgG (1:1000) for Ki67 and pSMAD2/3, Alexa Fluor 594 goat anti-rabbit IgG (1:1000) for F4/80 and PPAR-gamma. ProLong® Diamond Antifade Mountant with DAPI (P36971, Invitrogen, Carlsbad, CA, USA) was used to stain the nuclei and mount the samples. Sections incubated with conjugated primary antibodies were mounted without secondary antibody incubation. Fluorescent images were taken by Olympus Fluoview confocal microscope at 20×.

### Flow Cytometry

Flow cytometry was performed using single-cell suspensions obtained from skin and subcutaneous tissue. 1×1 cm tissue harvested from the mice hindlimb were minced with scissors and digested by using collagenase type I (1 mg/ml) and dispase (2 mg/ml) at 37° C. on a shaker. All suspensions were filtered through 100 μm filters.

Cells were then stained with following conjugated antibodies: APC-PDGFRα (1:100), APC-PDGFRβ (1:100), HCS LipidTOX red neutral lipid stains (1:200), APC-CD45 (1:150), PE-CD3 (1:150), PerCP-Cy5-CD4 (1:150). Flow cytometry was performed on the Beckman Cytoflex FCM using CytExpert software (Beckman Coulter, CA, USA) and data were analyzed with FlowJo software (Tree Star; Ashland, OR).

### Cell Culture and Rosiglitazone Treatment

Mice adipose-derived stem cells (ADSCs) were isolated from adult male C57BL/6J mice inguinal fat pad. ADSCs were cultured in DMEM/F12 (Sigma, Logan, UT) supplemented with 10% Fetal Bovine Serum (FBS) (Sigma) and antibiotics (penicillin and streptomycin, Hyclone). ADSCs were maintained at 37° C. under a 5% CO₂ atmosphere. For rosiglitazone treatment experiment, ADSCs were treated with/without recombinant TGFβ1 (10 ng/ml) (Invitrogen, USA) and rosiglitazone (10 μM) for 7 days after seeded (10× 10+ cells/ml) in 6-well plates.

### Real-Time PCR Analysis

Total RNA was isolated from ADSCs using TRI reagent and RNA extraction kit (Zymo Research, Irvine, CA). First-strand cDNA was synthesized by using iScript RT-PCR mix (BioRad, Hercules, CA) according to user manual. SYBR green qPCR master Mix (BioRad, Hercules, CA), primers, and cDNA were mixed in a final reaction volume of 10 μl. Real time polymerase chain reaction (RT-PCR) were performed by using Applied Biosystems 7300 Fast Real-Time PCR System (AB) according to the manufacturer's instructions. The oligo nucleotide primer purchased from Integrated DNA technology (IDT) were listed in Table 1.

### Statistical Analysis

Data are expressed as means±standard deviation. Statistically significant differences between two groups were established using student t-test. Statistical significance among three groups were established using one-way ANOVA. Significance set at a p-value<0.05. Data were analyzed and graphically presented using Prism (V9, GraphPad Software, La Jolla, CA, USA).

TABLE 1
Real time PCR Primer Sequences

| Gene name | Forward sequence | Reverse sequence |
|---|---|---|
| Adipoq | CCA CTT TCT CCT CAT TTC TG | CTA GCT CTT CAG TTG TAG TAA C |
| Col1a1 | GAT CTG TAT CTG CCA CAA TG | TGG TGA TAC GTA TTC CG |
| Ctgf | GAG GAA AAC ATT AAG GGC | AGA AAG CTC AAA CTT GAC AG |
| Fn1 | CCT ATA GGA TTG GAG ACA CG | GTT GGT AAA TAG CTG TTC GG |
| Lpl | GAG ACT CAG AAA AAG GTC | ATCGTC TTC AAA GAA CTC AGA TGC |
| Pdgfra | CTA GTT CCT GCA TCC ATT TTG | ATA TTT GAG ACA TTG CTG GC |
| Actb | GAT GTA TGA AGG CTT TGG TC | TGT GCA CTT TTA TTG GTC TC |

TABLE 2
Antibody information

| Antibody | Cat No. | Company |
|---|---|---|
| APC-PDGFRα | 135908 | BioLegend, San Diego, CA, USA |
| APC-PDGFRβ | 136008 | BioLegend, San Diego, CA, USA |
| Ki67 | AB9260 | EMD Millipore Corporation, |
| | | Temecula, CA, USA |
| FITC-CD4 | 100406 | BioLegend, San Diego, CA, USA |
| Alexa Fluor | 126612 | BioLegend, San Diego, CA, USA |
| 647-CD8b | | |
| F4/80 | NB110-40760 | Novus Biologicals, Centennial, |
| | | CO, USA |
| pSMAD2/3 | 8828S | Cell Signaling Technology, |
| | | Massachusetts, USA |
| PPAR-gamma | ABN1445 | EMD Millipore Corporation, |
| | | Temecula, CA, USA |
| APC-CD45 | 103112 | BioLegend, San Diego, CA, USA |
| PE-CD3 | 100206 | BioLegend, San Diego, CA, USA |
| PerCP-Cy5-CD4 | 100434 | BioLegend, San Diego, CA, USA |
| Alexa Fluor Plus | A32731 | Invitrogen, Carlsbad, CA, USA |
| 488 goat anti- | | |
| rabbit IgG | | |
| Alexa Fluor 594 | A32740 | Invitrogen, Carlsbad, CA, USA |
| goat anti-rabbit | | |
| IgG | | |

### REFERENCES FOR EXAMPLE 1

1. Dayan J H, Ly C L, Kataru R P, Mehrara B J. Lymphedema: Pathogenesis and Novel Therapies. Annual review of medicine. 2018; 69:263-276.
2. Meijer E F, Bouta E M, Mendonca C, et al. A retrospective analysis of commonly prescribed medications and the risk of developing breast cancer related lymphedema. Clin Res Trials. 2020; 6 (1).
3. Poulsen L, Kaur M, Jacobsen A L, et al. Comparison of upper extremity lymphedema after sentinel lymph node biopsy and axillary lymph node dissection: patient-reported outcomes in 3044 patients. Breast Cancer Res Treat. 2022; 191 (1): 87-96.
4. Slavin S A. Secondary Lymphedema. In: Greene A K, Slavin, S. A., Brorson H., ed. Lymphedema: Presentation, Diagnosis, and Treatment. Switzerland: Springer; 2015:79-95.
5. Ochalek K, Gradalski T, Szygula Z. Five-year assessment of maintenance combined physical therapy in postmastectomy lymphedema. Lymphatic research and biology. 2015; 13 (1): 54-58.
6. Coriddi M, Dayan J, Sobti N, et al. Systematic Review of Patient-Reported Outcomes following Surgical Treatment of Lymphedema. Cancers (Basel). 2020; 12 (3).
7. Bojinovic-Rodic D, Popovic-Petrovic S, Tomic S, Markez S, Zivanic D. Upper extremity function and quality of life in patients with breast cancer related lymphedema. Vojnosanitetski pregled. 2016; 73 (9): 825-830.
8. Teo I, Novy D M, Chang D W, Cox M G, Fingeret M C. Examining pain, body image, and depressive symptoms in patients with lymphedema secondary to breast cancer. Psycho-oncology. 2015; 24 (11): 1377-1383.
9. Weiss J, Daniel T. Validation of the Lymphedema Life Impact Scale (Llis): A Condition-Specific Measurement Tool for Persons with Lymphedema. Lymphology. 2015; 48 (3): 128-138.
10. Giuliano A E, Hunt K K, Ballman K V, et al. Axillary dissection vs no axillary dissection in women with invasive breast cancer and sentinel node metastasis: a randomized clinical trial. JAMA: the journal of the American Medical Association. 2011; 305 (6): 569-575.
11. Giuliano A E, Ballman K, McCall L, et al. Locoregional Recurrence After Sentinel Lymph Node Dissection With or Without Axillary Dissection in Patients With Sentinel Lymph Node Metastases: Long-term Follow-up From the American College of Surgeons Oncology Group (Alliance) ACOSOG Z0011 Randomized Trial. Annals of surgery. 2016; 264 (3): 413-420.
12. Giuliano A E, Ballman K V, McCall L, et al. Effect of Axillary Dissection vs No Axillary Dissection on 10-Year Overall Survival Among Women With Invasive Breast Cancer and Sentinel Node Metastasis: The ACOSOG Z0011 (Alliance) Randomized Clinical Trial. JAMA: the journal of the American Medical Association. 2017; 318 (10): 918-926.
13. Morton D L, Thompson J F, Cochran A J, et al. Final trial report of sentinel-node biopsy versus nodal observation in melanoma. The New England journal of medicine. 2014; 370 (7): 599-609.
14. Faries M B, Thompson J F, Cochran A J, et al. Completion Dissection or Observation for Sentinel-Node Metastasis in Melanoma. The New England journal of medicine. 2017; 376 (23): 2211-2222.
15. Brown J C, Cheville A L, Tchou J C, Harris S R, Schmitz K H. Prescription and adherence to lymphedema self-care modalities among women with breast cancer-related lymphedema. Support Care Cancer. 2014; 22 (1): 135-143.
16. Boyages J, Kastanias K, Koelmeyer L A, et al. Liposuction for Advanced Lymphedema: A Multidisciplinary Approach for Complete Reduction of Arm and Leg Swelling. Annals of surgical oncology. 2015; 22 Suppl 3: S1263-1270.
17. Greene A K, Voss S D, Maclellan R A. Liposuction for Swelling in Patients with Lymphedema. The New England journal of medicine. 2017; 377 (18): 1788-1789.
18. Hoffner M, Bagheri S, Hansson E, Manjer J, Troeng T, Brorson H. SF-36 Shows Increased Quality of Life Following Complete Reduction of Postmastectomy Lymphedema with Liposuction. Lymphatic research and biology. 2017; 15 (1): 87-98.
19. Hoffner M, Ohlin K, Svensson B, et al. Liposuction Gives Complete Reduction of Arm Lymphedema following Breast Cancer Treatment-A 5-year Prospective Study in 105 Patients without Recurrence. Plast Reconstr Surg Glob Open. 2018; 6 (8): e1912.
20. Schaverien M V, Munnoch D A, Brorson H. Liposuction Treatment of Lymphedema. Semin Plast Surg. 2018; 32 (1): 42-47.
21. Schaverien M V, Munro K J, Baker P A, Munnoch D A. Liposuction for chronic lymphoedema of the upper limb: 5 years of experience. Journal of plastic, reconstructive & aesthetic surgery: JPRAS. 2012; 65 (7): 935-942.
22. Lee D, Piller N, Hoffner M, Manjer J, Brorson H. Liposuction of Postmastectomy Arm Lymphedema Decreases the Incidence of Erysipelas. Lymphology. 2016; 49 (2): 85-92.
23. Lebovitz H E. Thiazolidinediones: the Forgotten Diabetes Medications. Curr Diab Rep. 2019; 19 (12): 151.
24. Nan Y M, Fu N, Wu W J, et al. Rosiglitazone prevents nutritional fibrosis and steatohepatitis in mice. Scand J Gastroenterol. 2009; 44 (3): 358-365.
25. Zhang H, You L, Zhao M. Rosiglitazone attenuates paraquat-induced lung fibrosis in rats in a PPAR gamma-dependent manner. Eur J Pharmacol. 2019; 851:133-143.
26. Wu M, Melichian D S, Chang E, Warner-Blankenship M, Ghosh A K, Varga J. Rosiglitazone abrogates bleomycin-induced scleroderma and blocks profibrotic responses through peroxisome proliferator-activated receptor-gamma. Am J Pathol. 2009; 174 (2): 519-533.
27. Iwayama T, Steele C, Yao L, et al. PDGFRalpha signaling drives adipose tissue fibrosis by targeting progenitor cell plasticity. Genes Dev. 2015; 29 (11): 1106-1119.
28. Joshi P A, Waterhouse P D, Kasaian K, et al. PDGFRalpha (+) stromal adipocyte progenitors transition into epithelial cells during lobulo-alveologenesis in the murine mammary gland. Nat Commun. 2019; 10 (1): 1760.
29. Wang Q A, Song A, Chen W, et al. Reversible De-differentiation of Mature White Adipocytes into Preadipocyte-like Precursors during Lactation. Cell Metab. 2018; 28 (2): 282-288 e283.
30. Johnson J A, Trasino S E, Ferrante A W, Jr., Vasselli J R. Prolonged decrease of adipocyte size after rosiglitazone treatment in high- and low-fat-fed rats. Obesity (Silver Spring). 2007; 15 (11): 2653-2663.

The citation of any document or reference is not to be construed as an admission that it is prior art with respect to the present invention.

### Example 2

Radiation therapy is a core component of adjuvant therapy for tumors involving the head/neck including bone, brain, and soft tissues. Of the over 80% of patients with head/neck cancer who receive radiation [Ref. 1], over 90% will develop tissue injury including atrophy, fibrosis, or tissue loss [Ref. 2-4] and up to 40% will develop partial or full-thickness skin loss or ulceration. These sequelae result in substantial morbidity including functional deficits, exposed bone or brain which lead to devastating infections, pain, and poor cosmesis [Ref. 5-8]. Unfortunately, reconstruction of these areas is challenging—thin skin grafts lead to an unacceptably high failure rate (30-100%) [Ref. 9,10].

Complex reconstructive operations, such as those performed by the PI, recruit skin and subcutaneous tissue to the radiated site, but require microvascular connections which demand long operative hours (10+ hours), technical expertise, and access to specialized operating supplies [Ref. 11-13]. As a result, strategies to alleviate radiation-induced tissue injury are needed to improve outcomes for cancer survivors after head/neck radiation.

### DETAILED DESCRIPTION OF SPECIFIC EMBODIMENTS

Supporting Literature. In the clinical setting, Bharatha et al found profound edema and reticulation in the subdermal adipose tissues after radiation to the head/neck region for cancer therapy using computed tomography (CT) [Ref. 14]. Consistent with these findings, Glastonbury et al reported atrophy in the subdermal tissues 8 weeks after head/neck region based on magnetic resonance imaging (MRI) [Ref. 15].

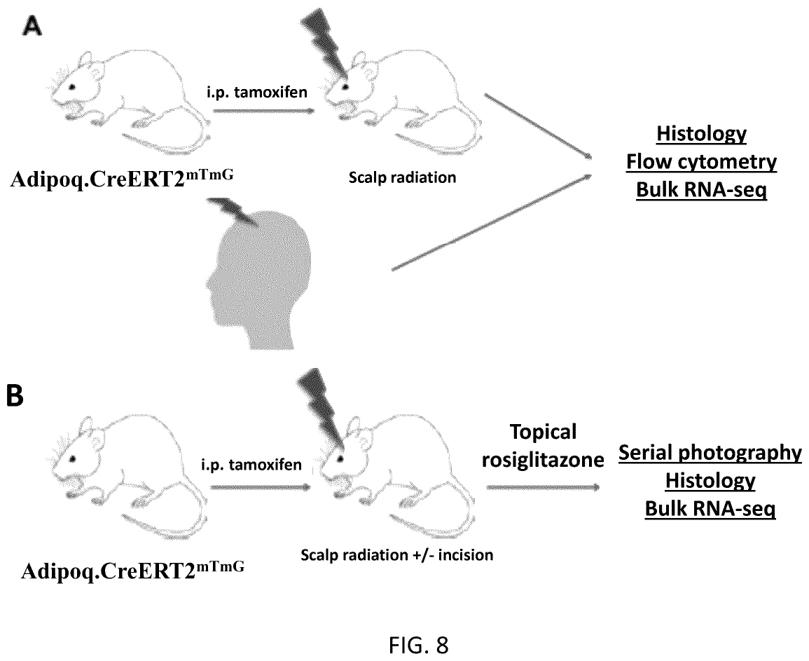

In a mouse model of total body irradiation, Poglio et al reported a significant reduction in inguinal fat pad mass within 7 days after radiation (0.23 v. 0.16 g, p<0.01) [Ref. 16]. They noted a significant and substantial reduction in diameter and number of adipocytes in the inguinal fat pad (0.58×10\hat{ }6 v. 0.32×10\hat{ }6, p<0.01) [Ref. 16]. Truong et al found that radiation directed to the mammary fat pad results in reduced adipogenic signaling based on reduced expression of Adipoq and increased fibrogenic signaling based on collagen expression [Ref. 17]. These findings provide strong evidence that radiation adversely impacts adipocytes in adipose depots (e.g., inguinal or mammary fat). However, Driskell et al found that dermal adipocytes are distinctly derived from dermal fibroblast progenitors, suggesting that these adipocytes are distinct from those found in adipose depots. Schmidt and Horsley showed that absence of dermal adipocytes led to poor wound re-epithelialization, disorganized collagen deposition, and reduced recruitment of regenerative ER− TR7+ fibroblasts and α-SMA+ myofibroblasts [Ref. 18]. Given these findings and the implicit differences between dermal adipocytes and adipocytes from depots, studies are required to determine how radiation impacts dermal adipocyte fate and function. The experimental strategy as disclosed herein and shown in FIG. 8 Panel A includes a control experiment using histology, flow cytometry, and bulk RNA sequencing following a radiation treatment of mouse or human scalp tissue, and a test of the effects of rosiglitazone (FIG. 8 Panel B) showing a mouse scalp treated with radiation and topical rosiglitazone, followed by histology and bulk RNA sequencing.

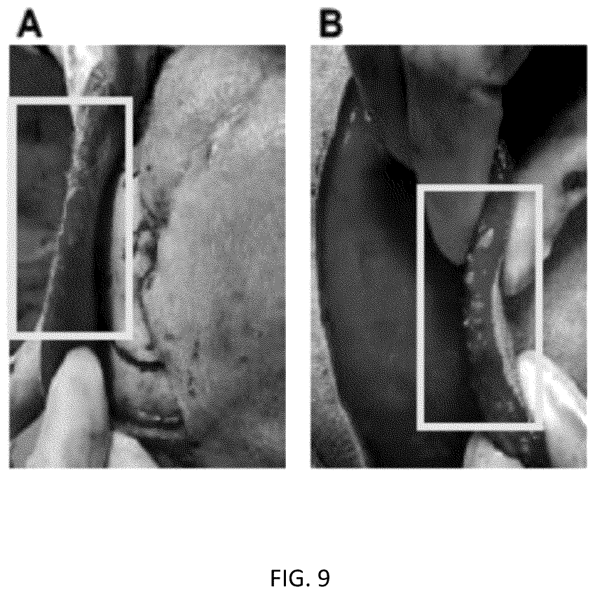

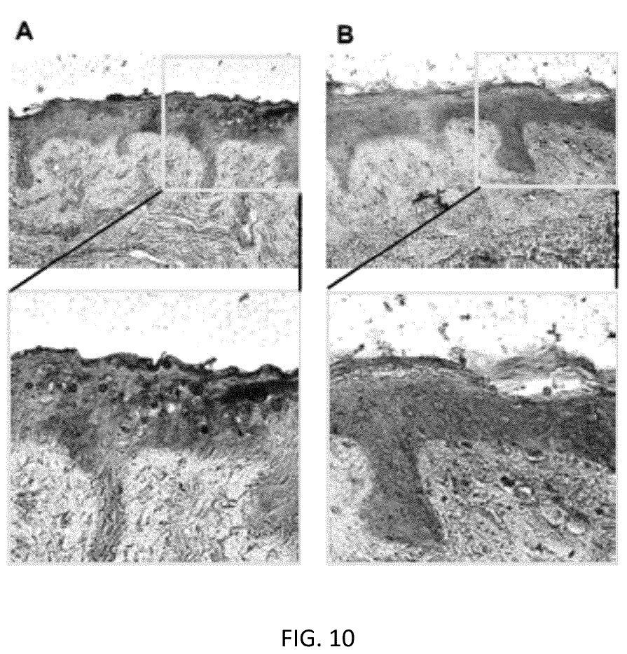

Supporting Preliminary Data. My lab has collected tissues from human irradiated scalps, with 6 samples available. Samples were obtained from patients with a history of radiation. Intraoperative evaluation of wounds verified paucity of subdermal adipose tissue in radiated scalp (FIG. 9A) when compared with the non-radiated scalp (FIG. 9 Panel B). We verified a relative reduction of adipocytes in the radiated scalp (FIG. 10 Panel A) based on Oil Red O staining with hematoxylin counterstain, when compared with non-radiated skin (FIG. 10. Panel B). However, further studies are required with our current tissue samples, and additional samples to obtained during this study period to quantify and evaluate the distribution of adipocytes.

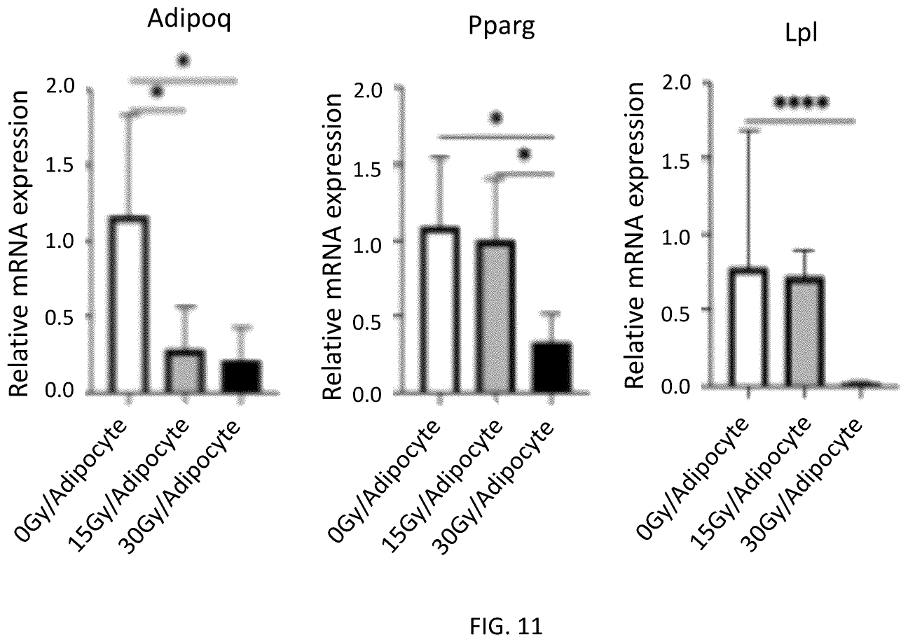

In the lab, adipocytes were isolated from human subdermal adipose tissue (Technical Preliminary Data) and treated with radiation (15 or 30 Gy) or no radiation (n=5/group). After culture for 5 days, mRNA was obtained and RT-qPCR performed (triplicate). We found reduced expression of adipogenic genes (Adipoq, Pparg, and Lpl) with 30 Gy radiation (FIG. 11, *p<0.05, ****p<0.001), providing in vitro evidence that radiation impairs the adipogenic profile of dermal adipocytes.

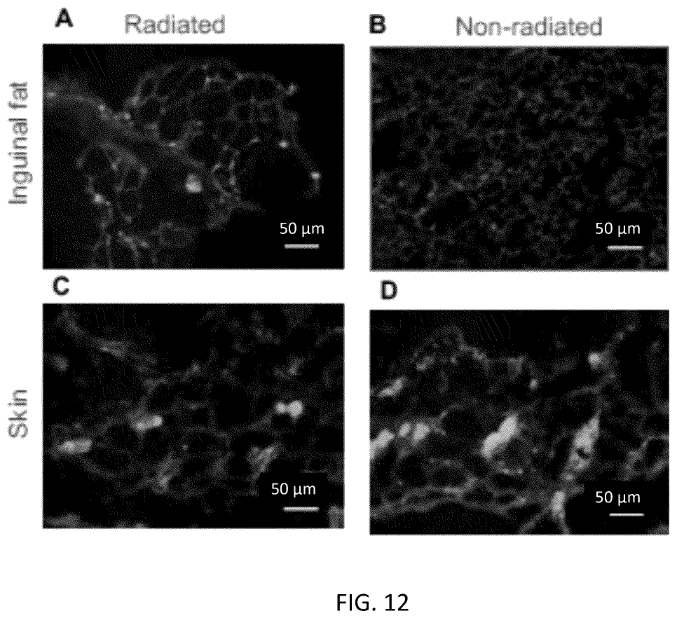

To examine how radiation affects dermal adipocytes in situ, inguinal fat or dorsal skin was excised from tamoxifen-treated adipocyte lineage-tracing mice (Adipoq.CreERT2^{mTmG}) and treated with radiation (30 Gy) or no radiation (n=6/group). Whole tissue culture was performed for 7 days followed by histologic evaluation. Our preliminary evaluation shows reduced adipocyte presence in the radiated inguinal fat (FIG. 12 Panel A) and radiated skin (FIG. 12. Panel C) relative to non-radiated inguinal fat (FIG. 12 Panel B) and non-radiated skin (FIG. 12 Panel D) respectively. Findings with inguinal fat consistent with previous literature [Ref. 16] validate this ex vivo strategy and preliminary data with skin.

Supporting Literature. Siebert et al reported an improvement in dorsal wound healing in obese mice (ob/ob) with systemic delivery of rosiglitazone, a PPARg agonist [Ref. 19]. They showed reduced time to healing of excisional cutaneous defects on the dorsum, and histologic evidence of improved re-epithelialization with rosiglitazone, when compared with untreated ob/ob mice. They also showed improved adipocyte organization within the healing wounds of rosiglitazone-treated mice. While promising, these findings must be interpreted in the context of the diabetic mouse model, as rosiglitazone is an anti-diabetic medication; therefore it is unclear whether the improvement in wound healing in obese mice was due to improvements in hyperglycemia, which is known to impede wound healing. Furthermore, given the adverse effects associated with rosiglitazone including fluid retention, heart failure, and fractures [Ref. 20,21], systemic rosiglitazone is unlikely to be used for radiation injury, a local phenomenon. Importantly, Scherer et al have shown that topical rosiglitazone indeed induces dermal adipose tissue hyperplasia localized to the area of treatment [Ref. 22]. However, it remains unclear whether this topical effect is sufficient to alleviate tissue injury after radiation.

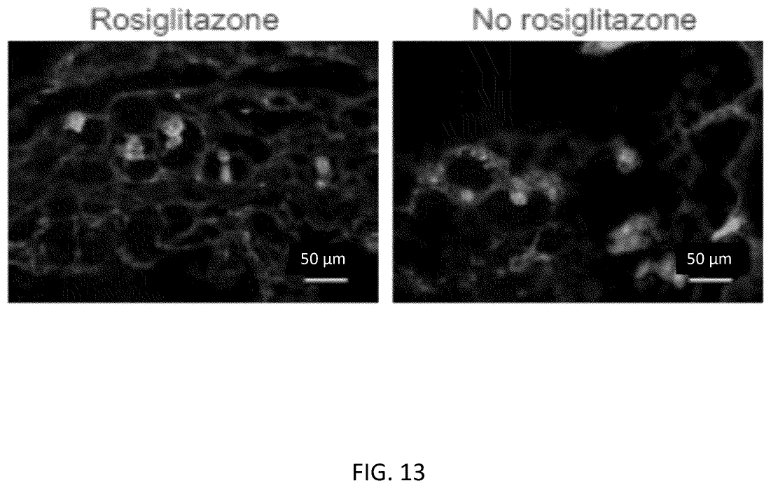

Supporting Preliminary Data. Based on our validated ex vivo radiation strategy (Significance), dorsal skin excised from tamoxifen-treated adipocyte lineage-tracing mice (Adipoq.CreERT2mTmG) was radiated (30 Gy) and cultured for 7 days with or without 10 μM rosiglitazone (n=6/group). Our preliminary evaluation verifies that rosiglitazone appeared to increase the histologic presence of adipocytes in radiated skin relative to no rosiglitazone control (FIG. 13).

### Innovation

The status quo as it pertains to radiation-induced tissue injury has largely focused on its deleterious impact on tissue vascularity. This proposal is innovative, because I seek to examine the novel premise that dermal adipocytes play a protective role against radiation-induced tissue injury using two strategies which present unique translational opportunities. Results from preliminary studies validate my examination of adipocyte fate after radiation.

I envision that augmented dermal adipogenesis at the irradiated site can improve healing after radiation. Findings from this study could lead to experiments to understand the underlying transcriptional identity of cell populations in the radiated field which undergo adipogenesis after rosiglitazone treatment. This is exciting because it could enable a strategy to improve radiated tissue with a topical treatment. To that end, a team of clinicians and scientific collaborators could be arranged to enable an investigator-initiated clinical trial studying topical rosiglitazone upon verification of efficacy in pre-clinical studies.

### Approach

We envision determining how radiation delivery modifies the fate of dermal adipocytes.

Introduction. Studies examining the detrimental effects of radiation have been largely focused endothelial dysfunction and its effect on tissue vascularity. Literature and preliminary data from my lab provide strong evidence that demonstrate that radiation impacts adipocyte fate (Significance). These findings are of importance given findings by Horsley and Schmidt demonstrating that dermal adipocytes orchestrate wound healing [Ref. 18]. The objective would be to identify how radiation impacts dermal adipocyte fate in vivo. Radiation will induce both fibrogenic and apoptotic profiles among mature adipocytes. My approach would be to irradiate the scalp of tamoxifen-treated adipocyte lineage-tracing mice (Adipoq.CreERT2^{mTmG}), followed by histologic evaluation of mG+ cells in the irradiated site for fibrogenic and apoptotic phenotypes and transcriptional profiles. The rationale is that successful completion will inform how radiation impacts a cell type which has been separately shown to orchestrate wound healing [Ref. 18].

Research Design. Adipocyte lineage-tracing mice (Adipoq.CreERT2^{mTmG}), obtained by breeding Adipoq. CreERT2 (Jackson Laboratory, strain 025124) and Rosa26^{mTmG} (strain 007676) mice, will be treated with i.p. tamoxifen on day-of-life 28 and 31. Adipocytes, defined by Adipoq expression, at the time of tamoxifen exposure, will undergo genetic recombination resulting in expression of membrane-bound GFP (mG+). Following a 2-wk washout period, mice will receive scalp radiation (30 Gy in 6 fractions) as previously described [Ref. 23,24]; controls will not receive radiation. Mice will be euthanized 48 hrs/1 wk/3 wks/9 wks after final radiation fraction. The scalp will be sectioned and stained to characterize cells derived from pre-irradiation mature adipocytes (mG+) with respect to proliferative (Ki67), apoptotic (TUNEL), fibrogenic (Col1, Acta2, Ctgf), and adipogenic (Adipoq, Lpl, Cebpa) phenotypes. Flow cytometry will be used to quantify these mG+ populations. FACS will be used to isolate mG+ cells and bulk RNA-seq performed to determine their aggregate transcriptional features with or without radiation.

Separately, radiated and non-radiated human scalp tissue will be evaluated histologically and via flow cytometry for adipocyte quantity and distribution.

Statistical Analysis. Male and female mice will be used. Same-gender and aggregate analyses will be performed. Experimental mice will receive radiation; controls will not. Mice will be euthanized 48 hrs./1 wk/3 wks/9 wks after final fraction delivery. To detect a 50% change in the quantity of Adipoq+ cells (adipocytes) in irradiated scalp with power of 0.8 and p<0.05, assuming s.d. of 30%, I will need 8 mice/group/timepoint/sex. To account for premature euthanization due to infection or failure to thrive, I will include 10 mice/group/timepoint/sex for histology. Flow cytometry will be performed with 5 mice/group/timepoint/sex. Bulk RNA-seq will be performed with n=5 mice/group/timepoint using FACS to isolate mG+ cells. RNA will be isolated and pooled total RNA will be used for library preparation. Bulk RNA-seq will be performed with Genewiz with bioinformatic analysis (see Vertebrate animals for full description) to identify gene sets with top scoring enrichment relative to non-radiated controls. Human samples from both sexes will be examined with 10 samples/group (radiated or non-radiated).

Histologic evaluation and quantification will be performed by two reviewers blinded to conditions. Data will be analyzed with Student T-test or Mann-Whitney test.

Expected Outcomes. I expect a significant increase in fibrogenic and apoptotic mG+ cells in the radiated scalp when compared with non-radiated scalp based on histologic evaluation and flow cytometry quantification. I expect increased expression of fibrogenic and apoptotic transcriptional profiles in mG+ cells from the radiated scalp, relative to mG+ cells from non-radiated scalp based on RNA-seq. I expect reduced presence of Adipoq+ or Oil Red O+ cells in the radiated scalp of patients when compared with the non-radiated scalp of patients.

Alternatives. If we do not note a difference in adipocyte quantity or fate in the irradiated mouse scalp when compared with controls using immunostaining or bulk RNA-seq, I would modify the fractionation schedule (30 Gy in single or two fractions), and increase total radiation dose (45 Gy). If I still do not note a difference, I would consider the alternative hypothesis that radiation does not induce a fibrogenic or apoptotic fate in dermal adipocytes in vivo. I would examine bulk RNA-seq data to identify alternative pathways which may be affected, as these alternative pathways could be responsible for observed changes in tissue quality. If I note a non-significant trend in changing adipocyte fate, I would increase the number of mice in the study to improve power. To determine whether the impact of radiation is long-lasting, I would perform experiments with longer time points. I would perform single-cell RNAseq with trajectory analysis [Ref. 25,26] to examine whether specific adipocyte subpopulations are likely to exhibit transcriptional changes upon radiation exposure.

We envision examining how augmented adipogenesis modifies cutaneous radiation injury.

Introduction. Previous studies have established that topical rosiglitazone, a PPARg agonist, augments dermal adipogenesis [Ref. 22]. Furthermore, systemic rosiglitazone improved wound healing in a diabetic mouse model and improved adipocyte organization within the wound site [Ref. 19]. However, systemic rosiglitazone has well-known adverse effects [Ref. 20,21], and it is unknown whether topical rosiglitazone may improve radiation-induced tissue injury. The objective would be to determine whether topical rosiglitazone augments dermal adipogenesis in radiated tissue, and alleviates cutaneous radiation injury. My working hypothesis is that topical rosiglitazone will augment adipogenesis in the radiated site and improve metrics of cutaneous radiation injury and incisional healing. My approach will be to irradiate the scalp of adipocyte lineage-tracing mice (Adipoq.CreERT2^{mTmG}) with or without creation of a 1-cm incision, followed by topical application of rosiglitazone to the scalp. Mice will be followed photographically for time to healing, and histologically for confirmation of adipocyte engraftment, tissue fibrosis, epithelialization, and collagen deposition and organization. We will also examine how rosiglitazone impacts the fate of mG+ and mT+ cells in the radiated site. The rationale is that successful completion will allow us to develop a topical formulation which can improve tissue quality after radiation.

Research Design. Examining the effect of topical rosiglitazone on radiation-induced tissue fibrosis. Adipoq. CreERT2^{mTmG} mice will be treated with tamoxifen followed by 2-week washout period; mice will receive scalp radiation (30 Gy in 6 fractions) [Ref. 23,24]. Experimental mice will receive topical rosiglitazone (5 μg in 50 mg CeraVe cream; total rosiglitazone: 0.1 mg) daily; controls will receive CeraVe cream alone [Ref. 22]. The radiated site will be monitored photographically for radiation-induced changes including hair loss and ulceration. Mice will be euthanized 48 hrs/1 wk/3 wks/9 wks after radiation. Early time points will elucidate whether rosiglitazone impacts inflammation. Tissue will be examined via flow cytometry and histology for presence and quantity of adipocytes, dermal fibrosis (picrosirius red), and up-regulation of local fibrogenic (Col1, Acta2, Ctgf, pSmad 2/3) and adipogenic (Adipoq, Lpl, Cebpa) signaling. We will evaluate whether adipocytes in the radiated site are derived from pre-radiation adipocytes (mG+) or non-adipocytes (mT+) to determine whether rosiglitazone imparts a direct protective effect on established adipocytes, or whether it induces adipogenic differentiation of non-adipocyte populations. FACS will be used to isolate mG+ and mT+ cells, and bulk RNA-seq performed to evaluate for rosiglitazone-mediated changes in adipogenic and fibrogenic transcriptional profiles.

Examining the effect of topical rosiglitazone on incisional healing in the radiated field. Tamoxifen-treated Adipoq. CreERT2^{mTmG} mice will receive radiation (30 Gy in 6 fractions) to the scalp [Ref. 23,24] followed by 1-cm cranio-caudal incision in the radiated field. Experimental mice will receive topical rosiglitazone (5 μg in 50 mg CeraVe cream; total rosiglitazone applied: 0.1 mg) daily; controls will receive CeraVe cream [Ref. 22]. Incisional healing will be monitored via photography. Mice will be euthanized 48 hrs/1 wk/3 wks/9 wks after radiation. Tissue will be examined for number and distribution of mG+ and mT+ adipocytes, collagen deposition and organization across the incision line, and epithelialization. Immunostaining for regenerative populations including ER-TR7+ fibroblasts and α-SMA+ myofibroblasts [Ref. 18] will be performed.

Statistical Analysis. Male and female will be used. We will require 10 mice/group/timepoint/sex for photographic evaluation and histology. Flow cytometry will be performed with 5 mice/group/timepoint/sex. RNA-seq will be performed with n=5 mice/group/timepoint; reproducibility and data analysis details for RNA-seq are in Statistical Analysis. Histologic evaluation will be performed by two reviewers blinded to conditions. Data will be analyzed with Student T-test or Mann-Whitney test.

Expected Outcomes. I expect reduced gross morphologic changes including hair loss and incidence and size of ulceration with topical rosiglitazone. I expect adipogenesis and reduced fibrosis and fibrogenic signaling in tissues treated with rosiglitazone. I expect that both mG+ and mT+ cells exhibit adipogenic signaling with rosiglitazone treatment, indicating that rosiglitazone preserves adipogenic features of mature adipocytes and induces adipogenic differentiation among non-adipocytes in the radiated field.

I expect reduced time to healing, increased epithelialization, increased collagen deposition across the incision line, and improved collagen organization with topical rosiglitazone. I also expect increased presence of fibroblasts and myofibroblasts along the incision line of rosiglitazone-treated tissues.

Alternatives. If we do not note increased adipogenesis with the topical rosiglitazone, we would first increase the topical dose. Alternative PPARg agonists such as pioglitazone, or non-glitazone pro-adipogenic agents such as FK614 [Ref. 27] may be considered. If we note a substantial number of mT+ (non-lineage marked) undergo adipogenesis with rosiglitazone, we would perform studies to examine the identity of these mT+ cells. If rosiglitazone reduces inflammation acutely, we will study whether delayed rosiglitazone improves adipogenesis, tissue quality, and incisional healing in sites with chronic radiation injury.

### REFERENCES FOR EXAMPLE 2

1. Strojan, P. et al. Treatment of late sequelae after radiotherapy for head and neck cancer. Cancer treatment reviews 59, 79-92, doi: 10.1016/j.ctrv.2017.07.003 (2017).
2. Stone, H. B., Coleman, C. N., Anscher, M. S. & McBride, W. H. Effects of radiation on normal tissue: consequences and mechanisms. The Lancet. Oncology 4, 529-536, doi: 10.1016/s1470-2045 (03) 01191-4 (2003).
3. Straub, J. M. et al. Radiation-induced fibrosis: mechanisms and implications for therapy. Journal of cancer research and clinical oncology 141, 1985-1994, doi: 10.1007/s00432-015-1974-6 (2015).
4. Manna, B. & Cooper, J. S. in StatPearls (2019).
5. Kies, M. S., Bennett, C. L. & Vokes, E. E. Locally advanced head and neck cancer. Current treatment options in oncology 2, 7-13, doi: 10.1007/s11864-001-0012-x (2001).
6 Baudelet, M. et al. Very late xerostomia, dysphagia, and neck fibrosis after head and neck radiotherapy. Head Neck 41, 3594-3603, doi: 10.1002/hed.25880 (2019).
7. Sroussi, H. Y. et al. Common oral complications of head and neck cancer radiation therapy: mucositis, infections, saliva change, fibrosis, sensory dysfunctions, dental caries, periodontal disease, and osteoradionecrosis. Cancer Med 6, 2918-2931, doi: 10.1002/cam4.1221 (2017)
8. Griffin, M. F., Drago, J., Almadori, A., Kalavrezos, N. & Butler, P. E. Evaluation of the efficacy of lipotransfer to manage radiation-induced fibrosis and volume defects in head and neck oncology. Head Neck 41, 3647-3655, doi: 10.1002/hed.25888 (2019).
9. Kurul, S., Dincer, M., Kizir, A., Uzunismail, A. & Darendeliler, E. Plastic surgery in irradiated areas: analysis of 200 consecutive cases. European journal of surgical oncology: the journal of the European Society of Surgical Oncology and the British Association of Surgical Oncology 23, 48-53, doi: 10.1016/s0748-7983 (97) 80142-4 (1997).
10. Senchenkov, A. et al. Outcomes of skin graft reconstructions with the use of Vacuum Assisted Closure (VAC®) dressing for irradiated extremity sarcoma defects. World journal of surgical oncology 5, 138, doi: 10.1186/1477-7819-5-138 (2007).
11. Harris, B. N. & Bewley, A. F. Minimizing free flap donor-site morbidity. Curr Opin Otolaryngol Head Neck Surg 24, 447-452, doi: 10.1097/MOO.0000000000000286 (2016).
12. Kansy, K. et al. Long-term donor site morbidity in head and neck cancer patients and its impact on quality of life: a cross-sectional study. Int J Oral Maxillofac Surg 48, 875-885, doi: 10.1016/j.ijom.2019.01.009 (2019).
13. Stalder, M. W. et al. Single Versus Simultaneous Double Free Flaps for Head and Neck Reconstruction: Comparison of Flap Outcomes and Donor-Site Morbidity. Ann Plast Surg 82, 184-189, doi: 10.1097/SAP.0000000000001713 (2019).
14. Bharatha, A., Yu, E., Symons, S. P. & Bartlett, E. S. Pictorial essay: early- and late-term effects of radiotherapy in head and neck imaging. Can Assoc Radiol J 63, 119-128, doi: 10.1016/j.carj.2010.09.004 (2012).
15. Glastonbury, C. M., Parker, E. E. & Hoang, J. K. The postradiation neck: evaluating response to treatment and recognizing complications. AJR Am J Roentgenol 195, W164-171, doi: 10.2214/AJR.09.4122 (2010).
16. Poglio, S. et al. Adipose tissue sensitivity to radiation exposure. Am J Pathol 174, 44-53, doi: 10.2353/ajpath.2009.080505 (2009).
17. Truong, J. L. et al. Creating a Favorable Microenvironment for Fat Grafting in a Novel Model of Radiation-Induced Mammary Fat Pad Fibrosis. Plast Reconstr Surg 145, 116-126, doi: 10.1097/PRS.0000000000006344 (2020).
18. Schmidt, B. A. & Horsley, V. Intradermal adipocytes mediate fibroblast recruitment during skin wound healing. Development 140, 1517-1527, doi: 10.1242/dev.087593 (2013).
19. Siebert, A., Goren, I., Pfeilschifter, J. & Frank, S. Anti-Inflammatory Effects of Rosiglitazone in Obesity-Impaired Wound Healing Depend on Adipocyte Differentiation. PLOS One 11, e0168562, doi: 10.1371/journal.pone.0168562 (2016).
20. Wallach, J. D. et al. Updating insights into rosiglitazone and cardiovascular risk through shared data: individual patient and summary level meta-analyses. BMJ 368, 17078, doi: 10.1136/bmj.17078 (2020).
21. Kahn, B. B. & McGraw, T. E. Rosiglitazone, PPARgamma, and type 2 diabetes. The New England journal of medicine 363, 2667-2669, doi: 10.1056/NEJMcibr1012075 (2010).
22. Zhang, Z. et al. Dermal adipose tissue has high plasticity and undergoes reversible dedifferentiation in mice. J Clin Invest 129, 5327-5342, doi: 10.1172/JCI130239 (2019).
23. Garza, R. M. et al. Studies in fat grafting: Part III. Fat grafting irradiated tissue—improved skin quality and decreased fat graft retention. Plast Reconstr Surg 134, 249-257, doi: 10.1097/PRS.0000000000000326 (2014).
24. Borrelli, M. R., Adem, S., Deleon Diaz, N. M., Nguyen, D., Momeni, A., Longaker, M. T., Wan, D. C. in Plastic Surgery Research Conference (Toronto, Ontario, CA, 2020).
25. Ramirez, A. K. et al. Single-cell transcriptional networks in differentiating preadipocytes suggest drivers associated with tissue heterogeneity. Nat Commun 11, 2117, doi: 10.1038/s41467-020-16019-9 (2020).
26. Saelens, W., Cannoodt, R., Todorov, H. & Saeys, Y. A comparison of single-cell trajectory inference methods. Nat Biotechnol 37, 547-554, doi: 10.1038/s41587-019-0071-9 (2019).
27. Minoura, H. et al. Mechanism by which a novel non-thiazolidinedione PPARg agonist, FK614, ameliorates insulin resistance in Zucker fatty rats. Diabetes Obes Metab 9, 369-378, doi: 10.1111/j.1463-1326.2006.00619.x (2007).

The citation of any document or reference is not to be construed as an admission that it is prior art with respect to the present invention.

Thus, the present invention provides devices and methods for treating secondary lymphedema of a subject.

In light of the principles and example embodiments described and illustrated herein, it will be recognized that the example embodiments can be modified in arrangement and detail without departing from such principles. Also, the foregoing discussion has focused on particular embodiments, but other configurations are also contemplated. In particular, even though expressions such as “in one embodiment”, “in an embodiment”, “in another embodiment”, “in other embodiments”, “in some embodiments”, or the like are used herein, these phrases are meant to generally reference embodiment possibilities, and are not intended to limit the invention to particular embodiment configurations. As used herein, these terms may reference the same or different embodiments that are combinable into other embodiments. As a rule, any embodiment referenced herein is freely combinable with any one or more of the other embodiments referenced herein, and any number of features of different embodiments are combinable with one another.

Although the invention has been described in considerable detail with reference to certain embodiments, one skilled in the art will appreciate that the present invention can be used in alternative embodiments to those described, which have been presented for purposes of illustration and not of limitation. Therefore, the scope of the appended claims should not be limited to the description of the embodiments contained herein.

1. A method for treating and/or preventing lymphedema in a subject, the method comprising:
a. administering to a subject in need of treatment and/or prevention of lymphedema an effective amount of a peroxisome proliferator-activated receptor gamma agonist (PPARg agonist) or a pharmaceutically acceptable salt thereof.

2. The method of claim 1, wherein the PPARg agonist is a thiazolidinedione or a pharmaceutically acceptable salt thereof.

3. The method of claim 2, wherein the thiazolidinedione is troglitazone, rosiglitazone or pioglitazone.

4. The method of claim 1, wherein the effective amount of the PPARg agonist is administered systemically.

5. The method of claim 1 further comprising co-administration of compression therapy.

6. The method of claim 1, wherein the effective amount of the PPARg agonist is administered systemically.

7. A method for reducing the total amount of fibroadipose tissue in a subdermal layer in a subject, the method comprising:
a. administering to the subject in need of reducing the total amount of fibroadipose tissue in the subdermal layer an effective amount of a PPARg agonist, wherein the PPARg agonist reduces fibrosis, reduces the number of adipocytes, and reduces the size of adipocytes.

8. The method of claim 7, wherein the PPARg agonist is a thiazolidinedione, or a pharmaceutically acceptable salt thereof.

9. The method of claim 8, wherein the thiazolidinedione is troglitazone, rosiglitazone, pioglitazone, or a pharmaceutically acceptable salt thereof.

10. The method of claim 7, wherein the effective amount of the PPARg agonist is administered systemically.

11. A method for rescuing adipogenic gene expression in a subject, the method comprising:
a. administering to the subject in need of rescuing adipogenic gene expression an effective amount of a PPARg agonist.

12. The method of claim 11, wherein the adipogenic gene expression was reduced among cells which have been exposed to tumor necrosis factor alpha (TNFα).

13. The method of claim 11, wherein the PPARg agonist is a thiazolidinedione.

14. The method of claim 12, wherein the thiazolidinedione is troglitazone, rosiglitazone or pioglitazone.

15. The method of claim 11, wherein the effective amount of the PPARg agonist is administered systemically.

16. A method for reducing fibrogenic gene expression in a subject, the method comprising:
a. administering to the subject in need of reduced fibrogenic gene expression an effective amount of a PPARg agonist.

17. The method of claim 16, wherein the PPARg agonist reduces fibrogenic gene expression by cells which have been exposed to transforming growth factor-beta 1 (TGFβ1).

18. The method of claim 17, wherein the PPARg agonist is a thiazolidinedione.

19. The method of claim 18, wherein the thiazolidinedione is troglitazone, rosiglitazone or pioglitazone.

20. The method of claim 16, wherein the effective amount of the PPARg agonist is administered systemically.

21. The method of claim 16, wherein the PPARg agonist is rosiglitazone administered orally at 4 mg/day, 5 mg/day, 6 mg/day, 7 mg/day or 8 mg/day.

22. A method for treating and/or preventing a radiation-induced skin tissue injury in a subject undergoing or having received radiation therapy, the method comprising:
a. administering topically to skin tissue of a subject in need of treatment and/or prevention of a radiation-induced skin tissue injury an effective amount of a PPARg agonist.

23. The method of claim 22, wherein the radiation-induced tissue injury is atrophy, fibrosis, or tissue loss, partial-thickness skin loss, full-thickness skin loss, ulceration or any combination thereof.

24. The method of claim 23, wherein the PPARg agonist is a thiazolidinedione.

25. The method of claim 24, wherein the thiazolidinedione is troglitazone, rosiglitazone or pioglitazone.

26. The method of claim 25, wherein rosiglitazone is administered topically as a mixture with a carrier, where the weight ratio of rosiglitazone to carrier is in a range of about 1:5,000 to about 1:20,000.

27. The method of claim 13, wherein the PPARg agonist is rosiglitazone administered orally at 4 mg/day, 5 mg/day, 6 mg/day, 7 mg/day or 8 mg/day.
